(19) United States
(12) Patent Application Publication
(10) Pub. No.: US 2008/0318939 A1
(43) Pub. Date: Dec. 25, 2008

(54) METHODS FOR TREATING OPHTHALMIC DISORDERS

(76) Inventors: Jeffrey P. WHITTEN, Santee, CA (US); Sean O'Brien, Carlsbad, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/143,256

(22) Filed: Jun. 20, 2008

Related U.S. Application Data
(60) Provisional application No. 60/945,878, filed on Jun. 22, 2007.

(51) Int. Cl. A61K 31/5383; A61P 27/00; A61K 31/496

(52) U.S. Cl. 514/229.5; 514/253.02

(57) ABSTRACT

The invention relates in part to methods for treating ophthalmic conditions using compounds described herein. The conditions to be treated include various ocular cancers. The compositions employed include a polycyclic quinolinone derivative or related heteroaromatic analog thereof.

### REFERENCE TO EARLIER APPLICATIONS

This application claims benefit of priority to U.S. Provisional Application Ser. No. 60/945,878, filed Jun. 22, 2007, which is incorporated herein by reference in its entirety.

### FIELD OF THE INVENTION

The invention relates in part to methods and compositions for treating biological disorders involving the eye.

### DISCLOSURE OF THE INVENTION

The present invention in part provides chemical compounds having certain biological activities that include, but are not limited to, inhibiting cell proliferation. Thus, compounds described herein can be utilized to treat ophthalmic conditions.

Compounds of the invention fall within the general formulae described hereafter. Certain compositions comprise a compound described herein in combination with eye cells. Certain compositions can be utilized to treat an ophthalmic condition by administering to a subject a therapeutic agent described herein in amount effective to treat the condition, whereby the condition is treated. These and other embodiments of the invention are described in the description that follows.

### MODES OF CARRYING OUT THE INVENTION

Therapeutic agents described herein can be utilized to treat ophthalmic conditions. As the therapeutic agents are useful for inhibiting cell proliferation, such compounds can be utilized to treat ophthalmic conditions associated with aberrant cell proliferation. The therapeutic agents can result in cell apoptosis and cell necrosis, and can specifically target proliferation of cells leading to an aberrant cell proliferation condition over “normal” cells. Examples of therapeutic agents and conditions that can be treated by the agents are described hereafter.

Therapeutic Agents

The invention in part provides methods for treating ophthalmic conditions, such as ophthalmic conditions associated with aberrant cell proliferation, by administering to a subject in need of such treatment a therapeutically effective amount of a therapeutic agent in an amount effective to treat the condition.

The therapeutic agent may be administered in combination with another agent, and the combination may be administered as separate pharmaceutical compositions or admixed in a single pharmaceutical composition. The therapeutic agent and the combination agent also may be administered separately, including at different times and with different frequencies, as long as the combination agent is administered at a time that increases the potency of the therapeutic agent. In some embodiments, the combination agent and the therapeutic agent are administered at the same time, whether in separate dosages or admixed in a single dosage. Where the frequency of administration of the two materials can be adjusted to match, the combination agent and therapeutic agent are preferably combined into a single pharmaceutical composition, so the treated patient may receive a single dosage (e.g., oral or injection), for example.

Therapeutic agents of the invention are compounds that inhibit cell proliferation. Certain therampeutic agents can inhibit RNA biosynthesis, and some can bind to certain motifs in nucleic acids. Therapeutic agents to be used can be selected from several different classes of compounds, such as those described below, and methods for making and using them are known in the art. Several preferred classes of these therapeutic agents are described below.

In one aspect, the therapeutic agent can be a compound of formula (TA1-1):

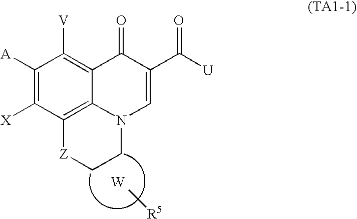

and pharmaceutically acceptable salts, esters and prodrugs thereof;

wherein V is H, halo, or NR¹R²;

A is H, fluoro, or NR¹₂;

Z is O, S, NR¹ or CH₂;

U is OR² or NR¹R²;

X is OR², NR¹R², halo, azido, or SR²;

n is 1-3;

wherein in NR¹R², R¹ and R² may form a double bond or a ring, each of which is optionally substituted;

R¹ is H or a C₁₋₆ alkyl;

R² is H or a C₁₋₁₀ alkyl or C₂₋₁₀ alkenyl optionally containing one or more non-adjacent heteroatoms selected from N, O, and S, and optionally substituted with a carbocyclic or heterocyclic ring; or R² is an optionally substituted heterocyclic ring, aryl or heteroaryl;

R⁵ is a substituent at any position on W; and is H, OR², C₁₋₆ alkyl, C₂₋₆ alkenyl, each optionally substituted by halo, ═O or one or more heteroatoms; or R⁵ is an inorganic substituent; and

W is an optionally substituted aryl or heteroaryl, which may be monocyclic or fused with a single or multiple ring and optionally containing a heteroatom;

or a compound having formula (TA1-2):

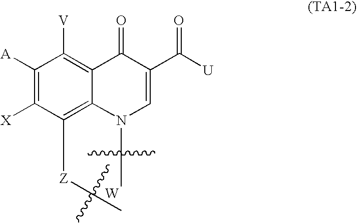

wherein V, A, X, Z and U are as defined in formula TA1-1, and W is selected from the group consisting of

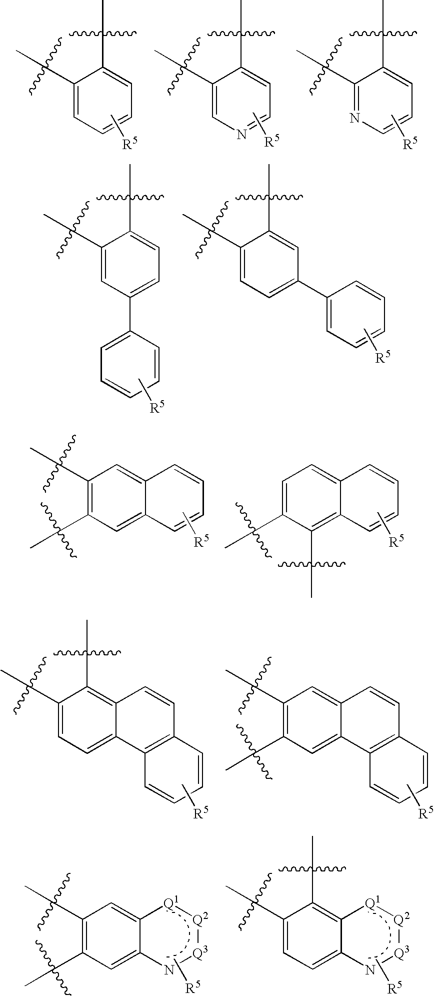

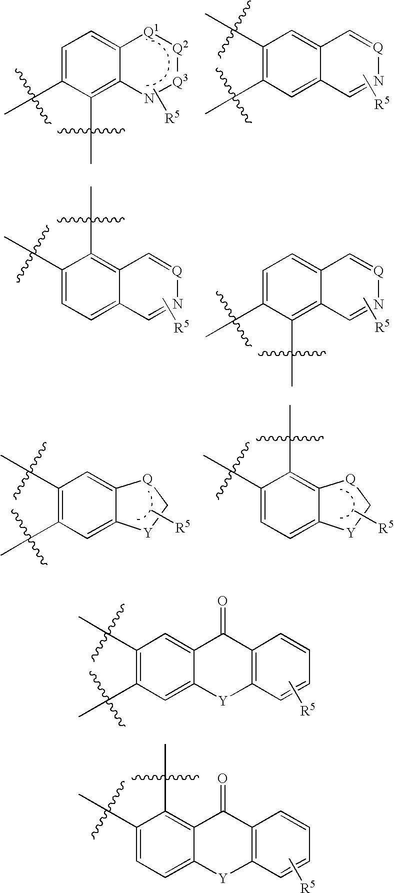

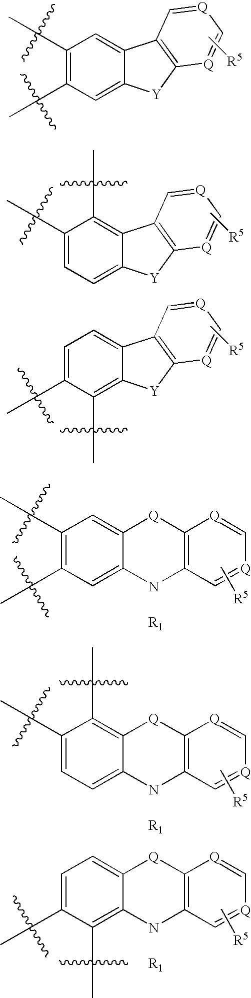

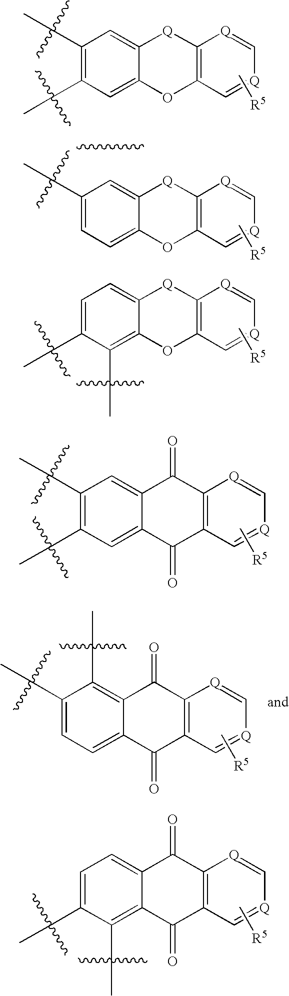

wherein Q, Q¹, Q², and Q³ are independently CH or N;

Y is independently O, CH, ═O or NR¹; and

R⁵ is as defined in formula 1.

Compounds of this structure, and methods for making and using them, are described in U.S. patent application Ser. No. 11/106,909, naming Whitten, et al., which is entitled SUBSTITUTED QUINOBENZOXAZINE ANALOGS AND METHODS OF USING THEREOF, and was filed on Apr. 15, 2005.

In a specific embodiment of the therapeutic agents of formula (TA1-1), the therapeutic agent is a compound having a structure of formula (TA1-1A):

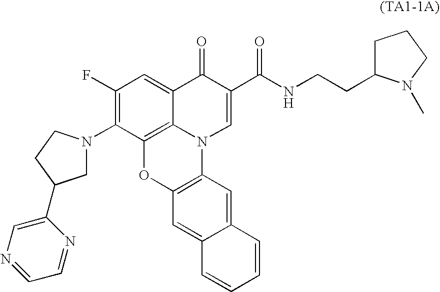

or a pharmaceutically acceptable salt, esters or prodrug thereof, or a specific isomer or mixture of isomers thereof. This compound can exist in multiple isomeric forms; in one embodiment, the compound of formula TA1-1A can be CX-3543, which is the isomer in which the pyrazine-substituted pyrrolidine (the five membered ring on the left side as drawn above) is racemic, while the pyrrolidine ring on the right side is one isomer, having the configuration as shown here:

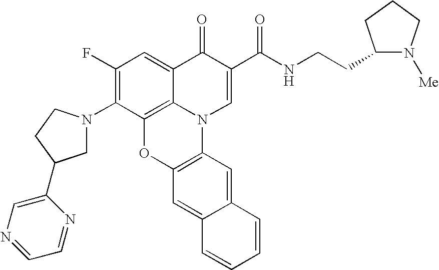

In another aspect, the therapeutic agent can be a compound having the general formula:

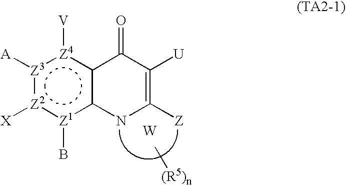

and pharmaceutically acceptable salts, esters and prodrugs thereof;

wherein B, X, A, or V is absent if Z¹, Z², Z³, or Z⁴ respectively is N , and independently H, halo, azido, R², CH₂R², SR², OR² or NR¹R² if Z¹, Z², Z³, or respectively is C; or

A and V, A and X, or X and B may form a carbocyclic ring, heterocyclic ring, aryl or heteroaryl, each of which may be optionally substituted and/or fused with a cyclic ring;

Z is O, S, NR¹, CH₂, or C═O;

Z¹, Z², Z³ and Z⁴ are C or N, provided any three N are non-adjacent;

W together with N and Z forms an optionally substituted 5- or 6-membered ring that is fused to an optionally substituted saturated or unsaturated ring; said saturated or unsaturated ring may contain a heteroatom and is monocyclic or fused with a single or multiple carbocyclic or heterocyclic rings;

U is SO₃R², SO₂NR¹R², SO₂NR¹NR¹R², SO₂NR¹OR², SO₂NR¹—(CR¹₂)ₙ—NR³R⁴ or SO₂NR¹NR¹—(CR¹₂)ₙ—NR³R⁴ or SO₂NR¹—O—(CR¹₂)ₙ—NR³R;

in each NR¹R², R¹ and R² together with N may form an optionally substituted ring;

in NR³R⁴, R³ and R⁴ together with N may form an optionally substituted ring; R¹ and R³ are independently H or C₁₋₆ alkyl;

each R² is H, or a C₁₋₁₀ alkyl or C₂₋₁₀ alkenyl each optionally substituted with a halogen, one or more non-adjacent heteroatoms, a carbocyclic ring, a heterocyclic ring, wherein each ring is aryl or heteroaryl and optionally substituted; or R² is an optionally substituted carbocyclic ring or heterocyclic ring, wherein each ring is aryl or heteroaryl;

R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;

each R⁵ is a substituent at any position on ring W; and is H, OR², amino, alkoxy, amido, halogen, cyano or an inorganic substituent; or R⁵ is C₁₋₆ alkyl, C₂₋₆ alkenyl, C₂₋₆ alkynyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring that may be fused to an additional optionally substituted carbocyclic or heterocyclic ring; and

n is 1-6.

In the above formula (TA2-1), B may be absent when Z¹ is N, or is H or a halogen when Z¹ is C.

In yet another embodiment, a therapeutic agent can have a structure of general formula (TA2-2) or (TA2-3):

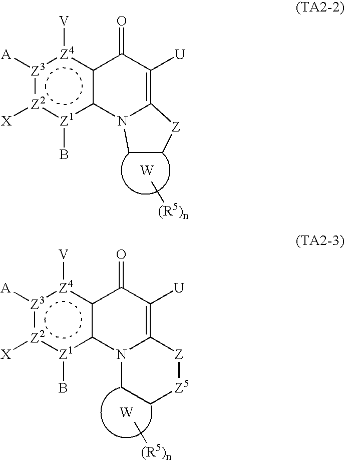

wherein V, A, X, B, W, U, Z, Z¹, Z², Z³, Z⁴ and n are as described above;
Z⁵ is O, NR¹, CR⁶, or C═O;
R⁶ is H, C₁₋₆ alkyl, hydroxyl, alkoxy, halo, amino or amido; and
Z and Z⁵ may optionally form a double bond.

In yet another embodiment, a therapeutic agent can have a structure of general formula (TA3-1) or (TA3-2):

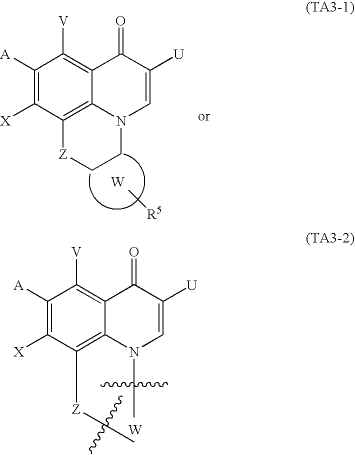

and pharmaceutically acceptable salts, esters and prodrugs thereof, where V, A, X, Z, W, U and R⁵ are previously described with respect to formulae TA2-1, TA2-2 and TA2-3.

In certain embodiments pertaining to compounds having structures of formulae TA2-1, TA2-2, TA2-3, TA3-1 and TA3-2, W together with N and Z in the above formula 1 or 3B forms an optionally substituted 5- or 6-membered ring that is fused to an optionally substituted aryl or heteroaryl ring selected from the group consisting of:

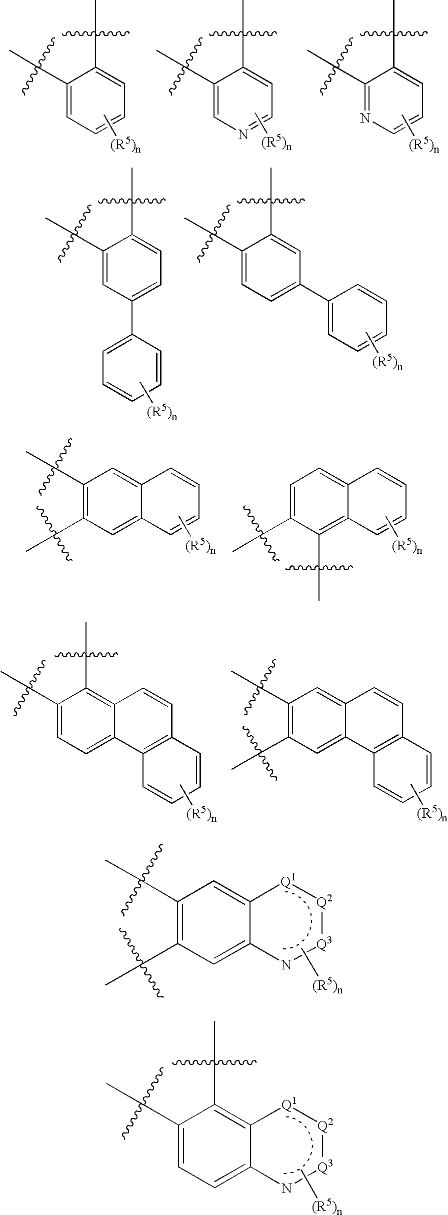

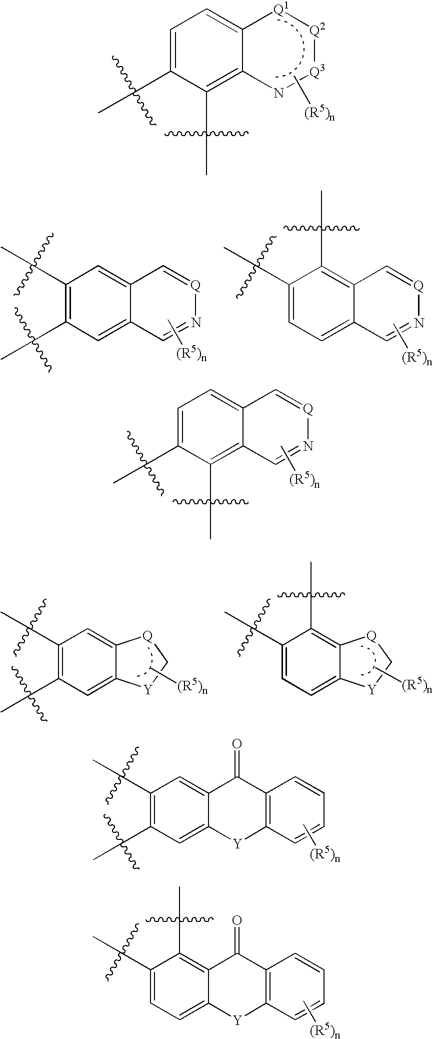

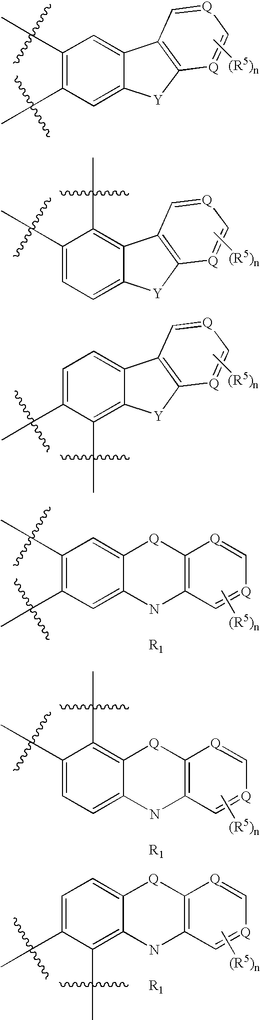

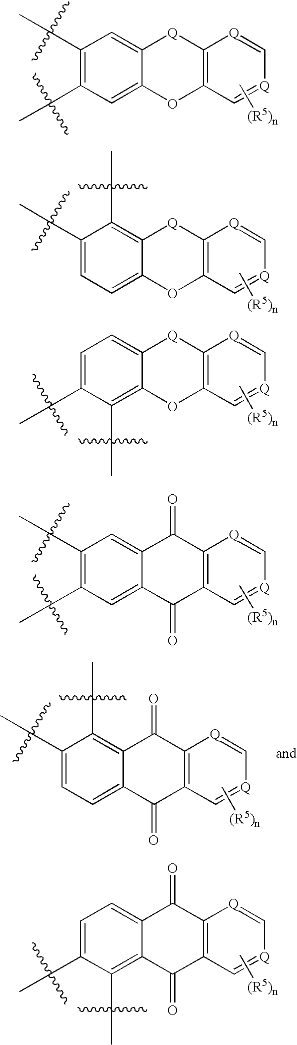

wherein each Q, Q¹, Q², and Q³ is independently CH or N;

Y is independently O, CH, C═O or NR¹;

n and R⁵ are as defined above.

In certain embodiments pertaining to compounds having structures of formulae TA2-1, TA2-2, TA2-3, TA3-1 and TA3-2, W together with N and Z may form a group having the formula selected from the group consisting of

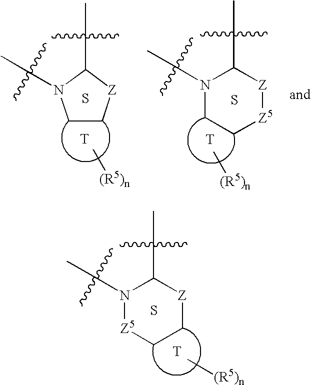

wherein Z is O, S, CR¹, NR¹, or C═O;
each Z⁵ is C(R⁶)₂, NR¹, or C═O, or Z and Z⁵ if adjacent can be —CR⁶═CR⁶— or —CR⁶═N—, and provided Z and Z⁵ if adjacent are not both NR¹;
each R¹ is H, C₁₋₆ alkyl, COR² or S(O)ₚR² wherein p is 1-2;
each R⁶ is independently H, or a substituent known in the art, including but not limited to hydroxyl, alkyl, alkoxy, halo, amino, or amido; and
ring S and ring T may be saturated or unsaturated.

In some embodiments pertaining to compounds having structures of formulae TA2-1, TA2-2, TA2-3, TA3-1 and TA3-2, W together with N and Z forms a 5- or 6-membered ring that is fused to a phenyl.

In certain embodiments pertaining to compounds having structures of formulae TA2-1, TA2-2, TA2-3, TA3-1 and TA3-2, U may be SO₂NR¹R² or SO₂NR¹OR² or SO₂NR¹NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S as ring members. For example, R² may be a C₁₋₁₀ alkyl substituted with an optionally substituted morpholine, thiomorpholine, imidazole, aminodithiadazole, pyrrolidine, piperazine, pyridine or piperidine. In other examples, R¹ and R² together with N form an optionally substituted piperidine, pyrrolidine, piperazine, morpholine, thiomorpholine, imidazole, or aminodithiazole. In some embodiments, U is SO₂NR¹R², and in some of these embodiments R¹ is H.

In some embodiments pertaining to compounds having structures of formulae TA2-1, TA2-2, TA2-3, TA3-1 and TA3-2, U may be SO₂NR¹—(CR¹₂)ₙ—NR³R⁴ or SO₂NR¹NR¹—(CR¹₂)ₙ—NR³R⁴ or SO₂NR¹O—(CR¹₂)ₙ—NR³R⁴; n is 1-4; and R³ and R⁴ in NR³R⁴ together form an optionally substituted piperidine, pyrrolidine, piperazine, morpholine, thiomorpholine, imidazole, or aminodithiazole. In some examples, U is SO₂NH—(CH₂)ₙ—NR³R⁴ wherein R³ and R⁴ together with N form an optionally substituted pyrrolidine, which may be linked to (CH₂)ₙ at any position in the pyrrolidine ring. In some embodiments, U is SO₂NR¹—(CR¹₂)ₙ—NR³R⁴, and in some of these embodiments R¹ is H. In one embodiment, R³ and R⁴ together with N form an N-methyl substituted pyrrolidine.

The preparation and activity of these compounds of formula (TA3-1) are described in International Patent Application No. PCT/US07/70794, filed Jun. 8, 2007, naming Nagasawa, et al., and entitled QUINOLONE ANALOGS DERIVATIZED WITH SULFONIC ACID, SULFONATE OR SULFONAMIDE.

In another aspect, the therapeutic agent is a compound of the following formula:

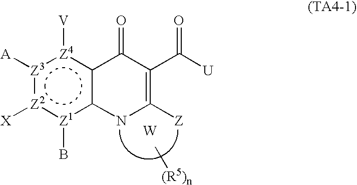

and pharmaceutically acceptable salts, esters and prodrugs thereof;

wherein B, X, A, or V is absent if Z², Z³, or Z⁴, respectively, is N, and independently H, halo, azido, R², CH₂R², SR², OR² or NR¹R² if Z², Z³, or Z⁴, respectively, is C; or

A and V, A and X, or X and B may form a carbocyclic ring, heterocyclic ring, aryl or heteroaryl, each of which may be optionally substituted and/or fused with a cyclic ring;

Z is O, S, NR¹, CH₂, or C═O;

Z¹, Z², Z³ and Z⁴ are C or N, provided any three N are non-adjacent;

W together with N and Z forms an optionally substituted 5- or 6-membered ring that is fused to an optionally substituted saturated or unsaturated ring; said saturated or unsaturated ring may contain a heteroatom and is monocyclic or fused with a single or multiple carbocyclic or heterocyclic rings;

U is R², OR², NR¹R², NR¹—(CR¹₂)ₙ—NR³R⁴, or N═CR¹R², wherein in N═CR¹R²R¹ and R² together with C may form a ring,

provided U is not H, and when U is OH, OR² or NH₂, then at least one of Z¹-Z⁴ is N;

in each NR¹R², R¹ and R² together with N may form an optionally substituted ring;

in NR³R⁴, R³ and R⁴ together with N may form an optionally substituted ring;
R¹ and R³ are independently H or C₁₋₆ alkyl;
each R² is H, or a C₁₋₁₀ alkyl or C₂₋₁₀ alkenyl each optionally substituted with a halogen, one or more non-adjacent heteroatoms, a carbocyclic ring, a heterocyclic ring, wherein each ring is aryl or heteroaryl and optionally substituted; or R² is an optionally substituted carbocyclic ring, heterocyclic ring, wherein each eing is aryl or heteroaryl;

R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;

each R is a substituent at any position on ring W; and is H, OR², amino, alkoxy, amido, halogen, cyano or an inorganic substituent; or R⁵ is C₁₋₆ alkyl, C₂₋₆ alkenyl, C₂₋₆ alkynyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring that may be fused to an additional optionally substituted carbocyclic or heterocyclic ring; and

n is 1-6.

In the above formula (TA4-1), B may be absent when Z¹ is N, or is H or a halogen when Z¹ is C.

In the above formula (TA4-1), W together with N and Z forms an optionally substituted 5- or 6-membered ring that is fused to an optionally substituted aryl or heteroaryl ring selected from the group consisting of:

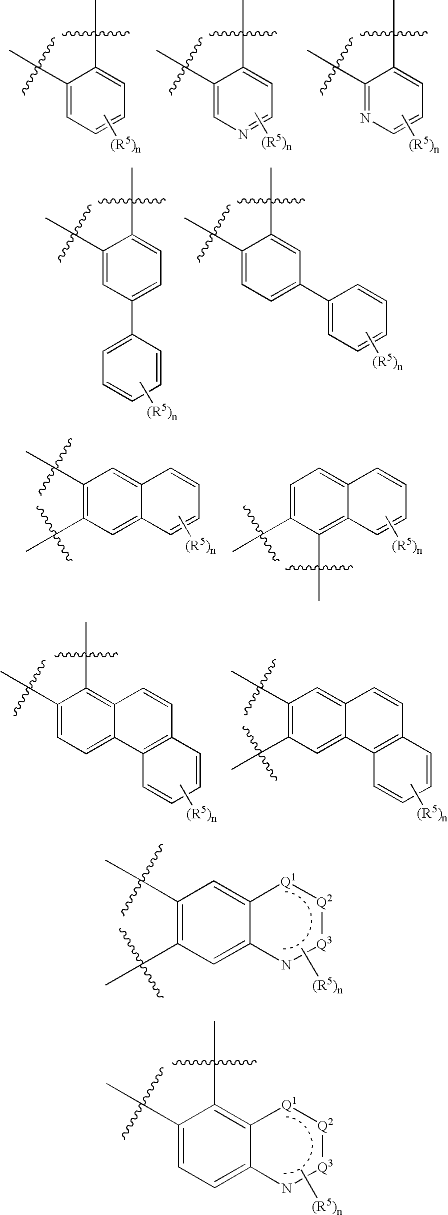

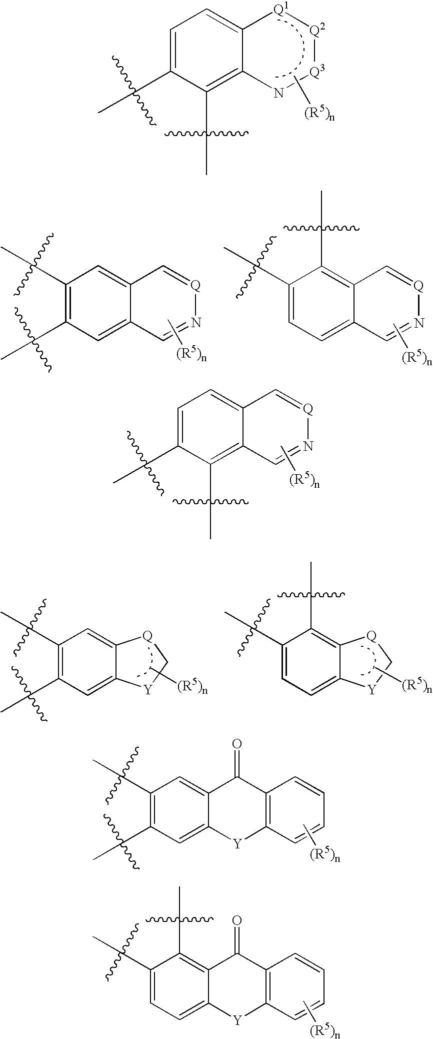

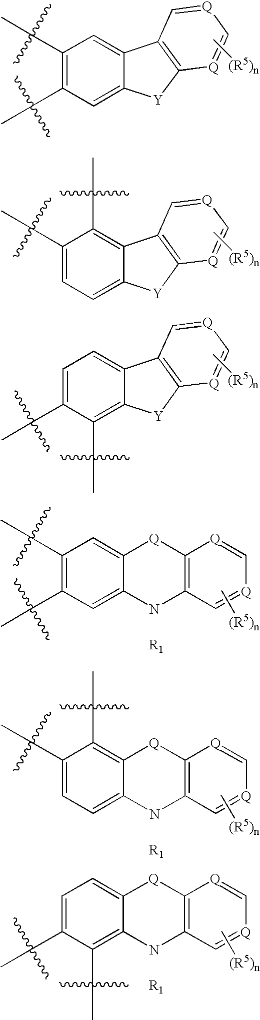

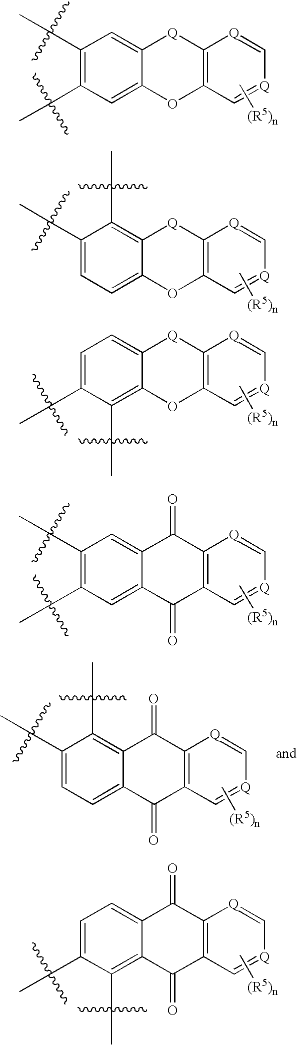

wherein each Q, Q¹, Q², and Q³ is independently CH or N;

Y is independently O, CH, C═O or NR¹;

n and R⁵ is as defined above.

In other embodiments, W together with N and Z form a group having the formula selected from the group consisting of

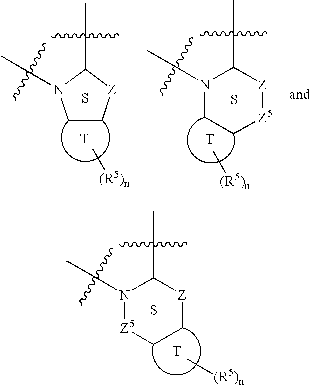

wherein Z is O, S, CR¹, NR¹, or C═O;

each Z⁵ is C(R⁶)₂, NR¹, or C═O, or Z and Z⁵ if adjacent can be —CR⁶═CR⁶— or —CR⁶═N—, a provided Z and Z⁵ if adjacent are not both NR¹;
each R¹ is H, C₁₋₆ alkyl, COR² or S(O)ₚR² wherein p is 1-2;
R⁶ is H, or a substituent known in the art, including but not limited to hydroxyl, alkyl, alkoxy, halo, amino, or amido; and
ring S and ring T may be saturated or unsaturated.

In some embodiments, W together with N and Z forms a 5- or 6-membered ring that is fused to a phenyl. In other embodiments, W together with N and Z forms a 5- or 6-membered ring that is optionally fused to another ring, when U is NR¹R², provided U is not NH₂. In certain embodiments, W together with N and Z forms a 5- or 6-membered ring that is not fused to another ring, when U is NR¹R² (e.g., NH₂).

In yet another embodiment, the compounds of the present invention have the general formula (TA4-2A) or (TA4-2B):

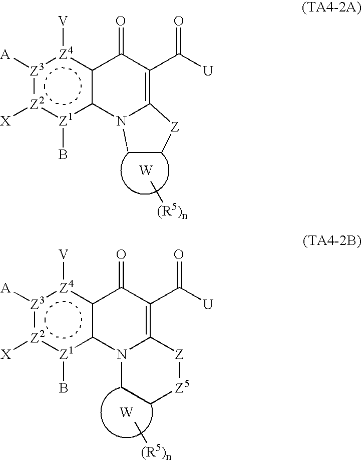

wherein A, B, V, X, U, Z, Z¹, Z², Z³, Z⁴ and n are as described for TA4-1;
Z⁵ is O, NR¹, CR⁶, or C═O;
R⁶ is H, C₁₋₆ alkyl, hydroxyl, alkoxy, halo, amino or amido; and
Z and Z⁵ may optionally form a double bond.

In the above formula (TA4-1), (TA4-2A) and (TA4-2B), U may be NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S as a ring member. For example, R² may be a C₁₋₁₀ alkyl substituted with an optionally substituted morpholine, thiomorpholine, imidazole, aminodithiadazole, pyrrolidine, piperazine, pyridine or piperidine. In other examples, R¹ and R² together with N form an optionally substituted piperidine, pyrrolidine, piperazine, morpholine, thiomorpholine, imidazole, or aminodithiazole.

The compounds of formula (TA4-1), and methods of making and using them, are described in U.S. patent application Ser. No. 11/228,636, naming Whitten, et al., entitled QUINOLONE ANALOGS, and filed on Sep. 16, 2005. An example of a compound that significantly permeates the blood-brain barrier has a structure of the following general formula (TA4-1 A):

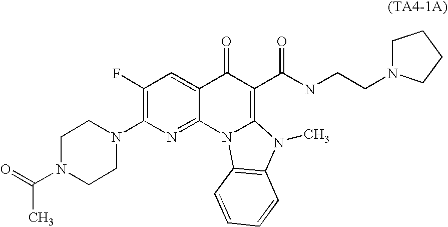

In yet another aspect, the therapeutic agent can be selected from compounds having this formula:

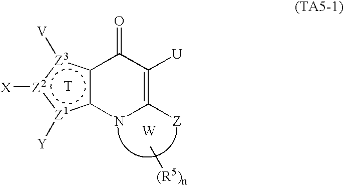

and pharmaceutically acceptable salts, esters and prodrugs thereof;

wherein V, X, and Y are absent if attached to a heteroatom other than Nitrogen, and independently H, halo, azido, R², CH₂R², SR², OR² or NR¹R² when attached to C or N; or

wherein V and X, or X and Y may form a carbocyclic ring, heterocyclic ring, aryl or heteroaryl, each of which may be optionally substituted and/or fused with a cyclic ring;

Z¹, Z² and Z³ are C, N, O or S, wherein among Z¹, Z² and Z³ there is at most one O atom, among Z¹, Z² and Z³ there is at most one S atom, and among Z¹, Z² and Z³ there is at most two carbon atoms;

Z is O, S, NR², CH₂ or C═O;

W together with N and Z forms an optionally substituted 5- or 6-membered ring that is fused to an optionally substituted aryl or heteroaryl, wherein said aryl or heteroaryl may be monocyclic or fused with a single or multiple ring, and wherein said ring optionally contains a heteroatom;

U is —C(═O)R, —COOR , —CONR¹R², —CONR¹—(CR¹₂)ₙ—NR³R⁴, SO₃R², SO₂NR¹R², SO₂NR¹NR¹R², SO₂NR¹OR², SO₂NR¹—(CR¹₂)ₙ—NR³R⁴ or SO₂NR¹NR¹—(CR¹₂)ₙ—NR³R⁴ or SO₂NR¹—O—(CR¹₂)ₙ—NR³R;

wherein in each NR¹R², R¹ and R² together with N may form an optionally substituted ring;

in NR³R⁴, R³ and R⁴ together with N may form an optionally substituted ring;

R¹ and R³ are independently H or C₁₋₆ alkyl;

each R² is H, or a C₁₋₁₀ alkyl or C₂₋₁₀ alkenyl each optionally substituted with a halogen, one or more non-adjacent heteroatoms selected from N, O and S, a carbocyclic ring, a heterocyclic ring, aryl or heteroaryl, wherein each ring is optionally substituted; or R² is an optionally substituted carbocyclic ring or heterocyclic ring, aryl or heteroaryl; or R² is COR¹ or S(O)ₓR¹ wherein x is 1-2;

R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;

each R⁵ is a substituent at any position on W; and is H, OR², amino, alkoxy, amido, halogen, cyano or an inorganic substituent; or R⁵ is C₁₋₆ alkyl, C₂₋₆ alkenyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring, optionally fused to an additional optionally substituted carbocyclic or heterocyclic ring; and

n is 1-6.

In the above formula (TA5-1), the ring labeled “T” is a five membered ring that can contain up to three heteroatoms selected from N, O, and S. Substituents V, X, and Y are as defined above, and each of them may be absent when the ring atom to which it is connected has no available open valence for substitution. The dashed circle indicates that each ring atom of ring T has a pi bond, which may be provided by either a heteroatom or an sp² hybridized carbon. In many embodiments, T is an aromatic ring, and in certain embodiments, T can be a non-aromatic ring. Ring “T” may, in some embodiments, form an optionally substituted 5-membered ring selected from the group consisting of:

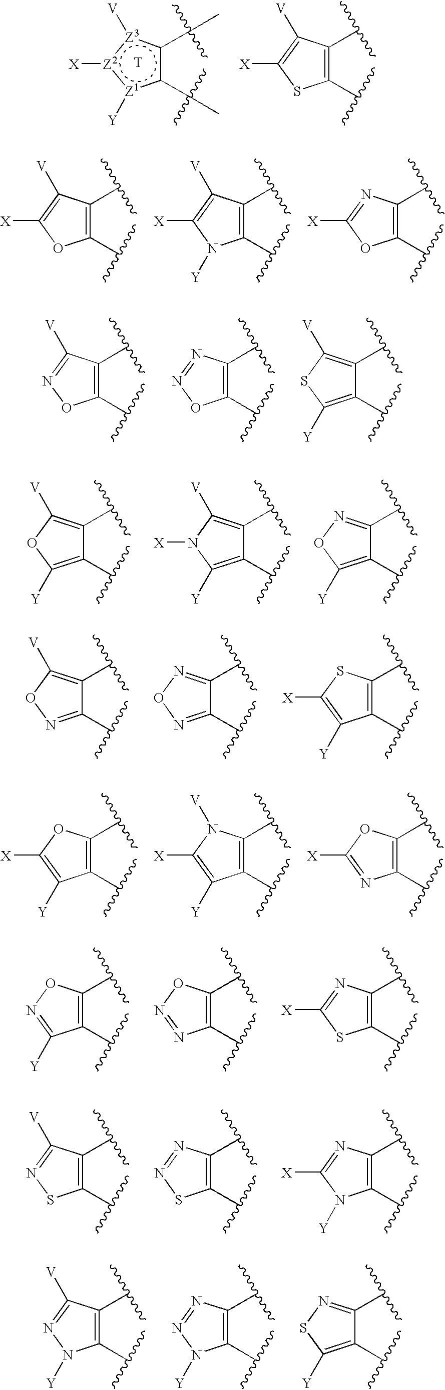

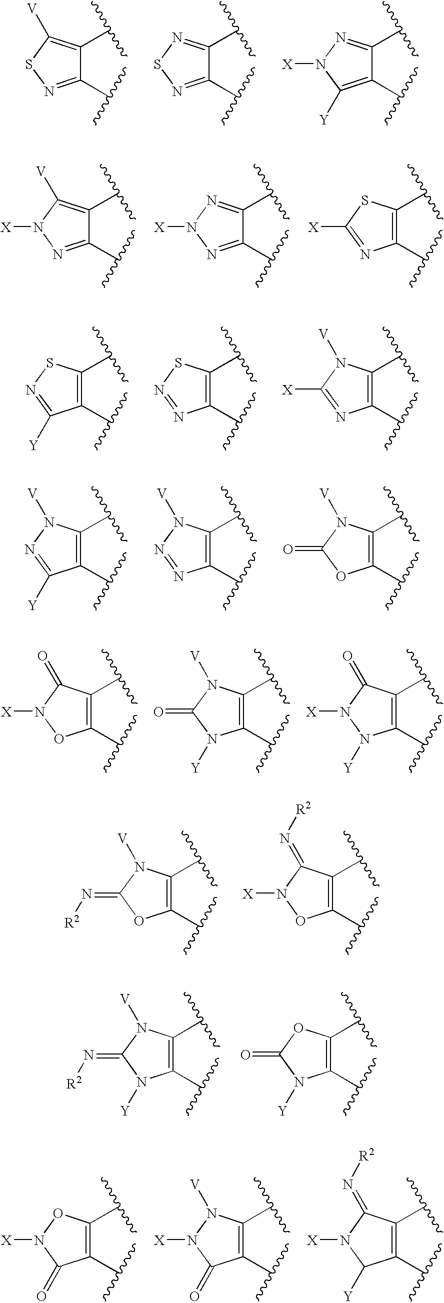

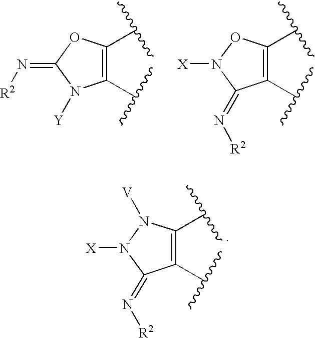

In the above formula (TA5-1), W together with N and Z may form an optionally substituted 5- or 6-membered aryl or heteroaryl ring that is fused to an optionally substituted aryl or heteroaryl selected from the group consisting of:

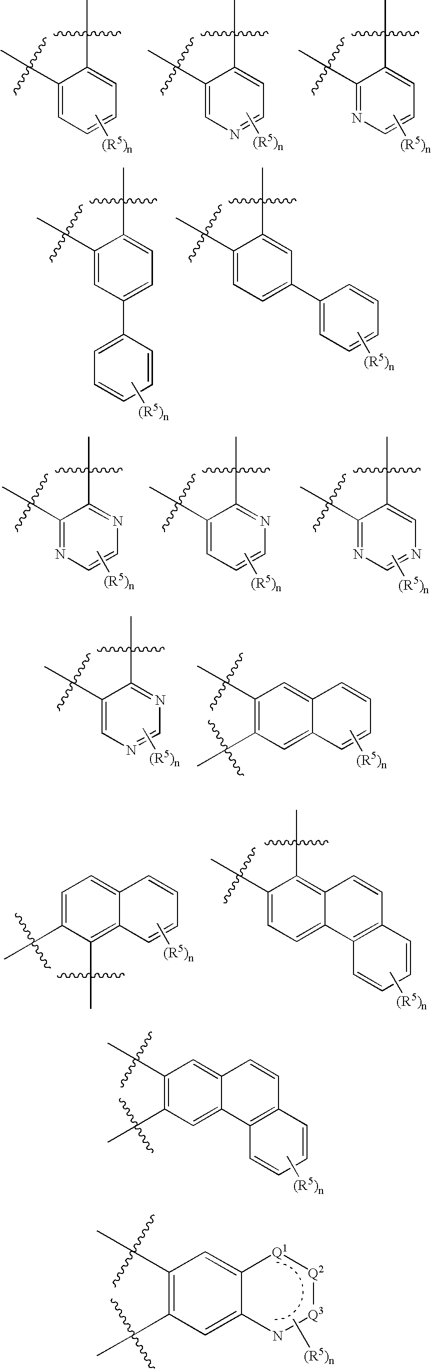

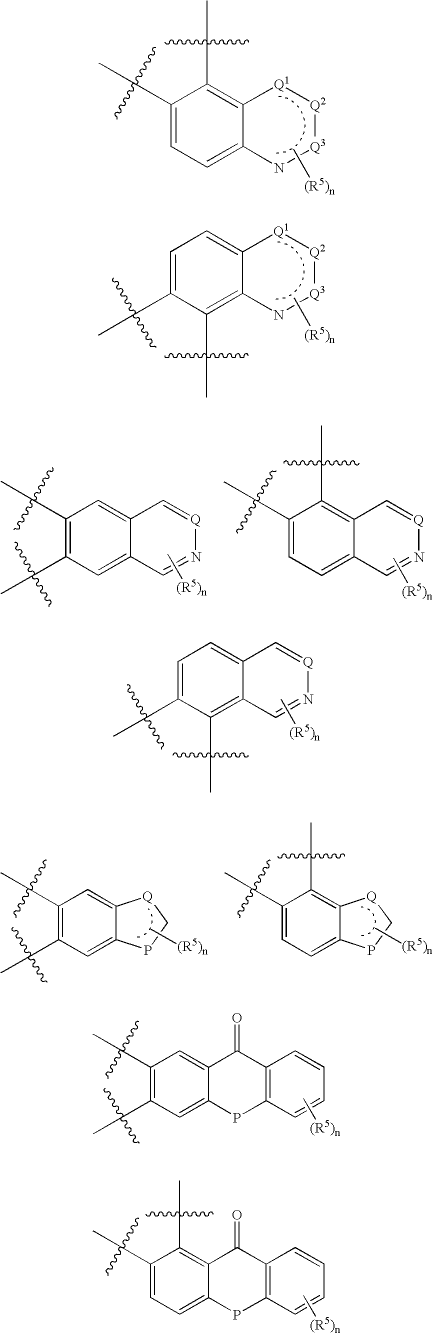

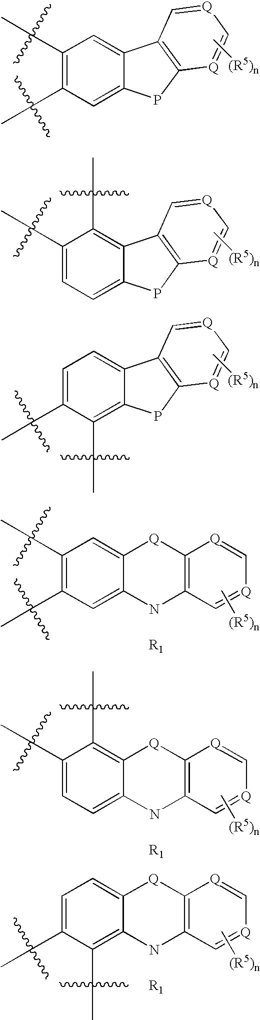

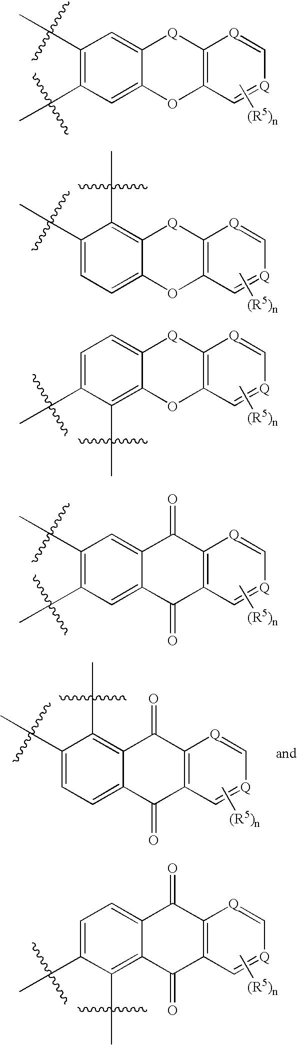

wherein each Q, Q¹, Q², and Q³ is independently CH or N;

P is independently O, CH, C═O or NR¹;

n and R⁵ are as defined above.

In other embodiments of these compounds, W together with N and Z may form a group having the formula selected from the group consisting of

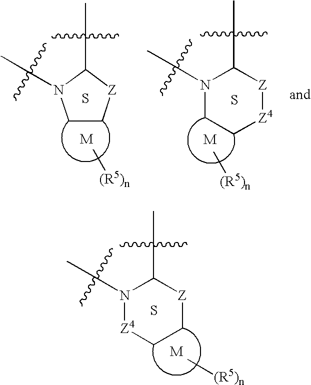

wherein Z is O, S, NR², CH₂ or C═O; each Z⁴ is C(R⁶)₂, NR¹, or C═O, or Z and Z⁴ if adjacent can be —CR⁶═CR⁶— or —CR⁶═N—, and provided Z and Z⁴ if adjacent are not both NR¹;

R⁶ is H, or a substituent known in the art, including but not limited to hydroxyl, alkyl, alkoxy, halo, amino, or amido; and

Ring S and M may be saturated or unsaturated.

In some embodiments, W together with N and Z may form a 5- or 6-membered ring that is fused to a phenyl.

In yet another embodiment, the compounds of the present invention have the general formula (TA5-2A) or (TA5-2B):

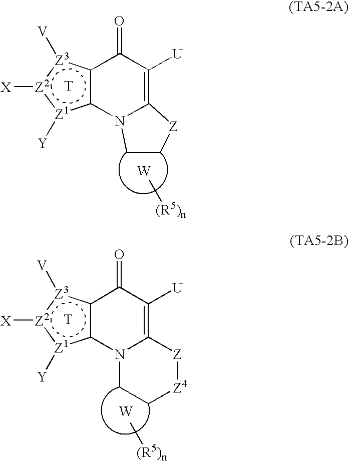

wherein U, V, W, X, Y, Z, Z¹, Z², Z³, R⁵ and n are as described above for TA5-1;

Z is CR⁶, NR⁶, or C═O; and

Z and Z⁴ may optionally form a double bond.

In the above formula (TA5-1), (TA5-2A) and (TA5-2B), U may be SO₂NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S. For example, R₂ may be a C₁₋₁₀ alkyl substituted with an optionally substituted morpholine, thiomorpholine, imidazole, aminodithiadazole, pyrrolidine, piperazine, pyridine or piperidine. In other examples, R¹ and R² together with N form an optionally substituted piperidine, pyrrolidine, piperazine, morpholine, thiomorpholine, imidazole, or aminodithiazole.

In other embodiments of these compounds, U is SO₂NR¹—(CR¹₂)ₙ—NR³R⁴; n is 1-4; each R¹ is H or alkyl; and R³ and R⁴ in NR³R⁴ together form an optionally substituted piperidine, pyrrolidine, piperazine, morpholine, thiomorpholine, imidazole, or aminodithiazole. In some examples, U is SO₂NH—(CH₂)ₙ—NR³R⁴ wherein R³ and R⁴ together with N form an optionally substituted pyrrolidine, which may be linked to (CH₂)ₙ at any position in the pyrrolidine ring. In one embodiment, R³ and R⁴ together with N form an N-methyl substituted pyrrolidine.

In one embodiment, the present invention provides compounds having formula (TA5-1), (TA5-2A) or (TA5-2B), wherein:

each of V and Y if present is independently H or halogen (e.g., chloro or fluoro);

X is —(R⁵)R¹R², wherein R⁵ is C or N and wherein in each —(R⁵)R¹R², R¹ and R² together may form an optionally substituted aryl or heteroaryl ring;

Z is NH or N-alkyl (e.g., N—CH₃);

W together with N and Z forms an optionally substituted 5- or 6-membered ring that is fused with an optionally substituted aryl or heteroaryl ring; and

U is —SO₂R⁵R⁶—(CH₂)ₙ—CHR²—NR³R⁴, wherein R⁵ is CR¹ or N; R¹ is H or alkyl; R⁶ is H or C₁₋₁₀ alkyl and wherein in the —CHR²—NR³R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In another embodiment, the present invention provides compounds having formula (TA5-1), (TA5-2A) or (TA5-2B), wherein:

V and Y if present is H or halogen (e.g., chloro or fluoro);

X if present is —(CR¹)R¹R² or NR¹R², wherein R¹ and R² together may form an optionally substituted aryl or heteroaryl ring;

Z is NH or N-alkyl (e.g., N—CH₃);

W together with N and Z forms an optionally substituted 5- or 6-membered ring that is fused with an optionally substituted aryl or heteroaryl ring; and

U is —SO₂NR⁶—(CH₂)ₙ—CHR²—NRR⁴, or —SO₂CR¹R⁶—(CH₂)ₙ—CHR²—NR³R⁴;

R⁶ is H or alkyl and wherein in the —CHR²—NR³ R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In yet another embodiment, the compounds of the present invention have the general formula (TA5-3):

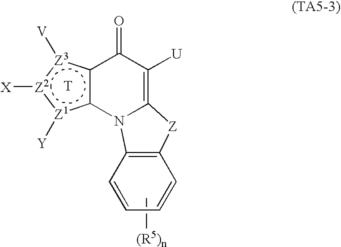

wherein U, V, X, Y, Z, Z¹, Z², Z³, R⁵ and n are as described above.

In yet another embodiment, the compounds of the present invention have the general formula (TA5-4A) or (TA5-4B):

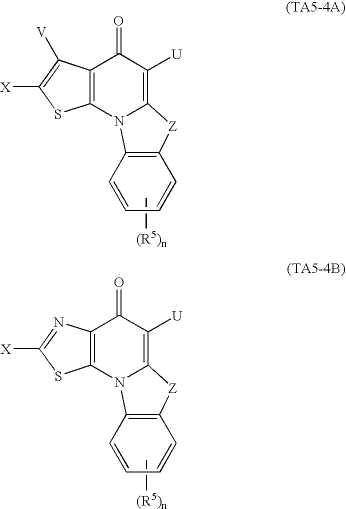

wherein U, V, X, Z, R⁵ and n are as described above for TA5-1.

Compounds of Formula (TA5-1), and methods for making and using them, are described in International Patent Application No. PCT/US07/70774, filed Jun. 8, 2007, naming Pierre, et al., and entitled PYRIDINONE ANALOGS.

In still another aspect, the therapeutic agent for the combinations of the invention can be a compound of the formula:

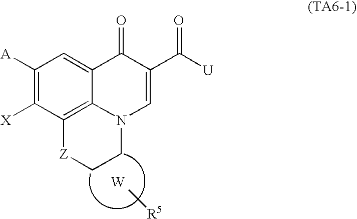

and pharmaceutically acceptable salts, esters and prodrugs thereof,

wherein X is H, OR², NR¹R², halogen, azido, SR² or CH₂R;

A is H, halogen, NR¹R², SR², OR², CH₂R², azido or NR¹—(CR¹₂)ₙ—NR³R⁴;

Z is O, S, NR or CH₂;

U is R², OR², NR¹R² or NR¹—(CR¹₂)ₙ—NR³R⁴ provided U is not H;

W is an optionally substituted aryl or heteroaryl, which may be monocyclic or fused with a single or multiple ring optionally containing a heteroatom;

wherein R¹ and R² together with N in NR¹R², and R³ and R⁴ together with N in NR³R⁴ may independently form an optionally substituted 5-6 membered ring containing N, and optionally O or S;

R¹ and R³ are independently H or a C₁₋₆ alkyl; and

R² and R⁴ are independently H, or a C₁₋₁₀ alkyl or C₂₋₁₀ alkenyl optionally containing one or more non-adjacent heteroatoms selected from N, O, and S, and optionally substituted with a substituted or unsubstituted aryl, heteroaryl, carbocyclic, or heterocyclic ring; or R² is an optionally cycloalkyl, substituted heterocyclic ring, aryl or heteroaryl;

R⁵ is a substituent at any position of W and is H, halo, cyano, azido, —CONHR¹, OR², or C₁₋₆ alkyl or C₂₋₆ alkenyl, each optionally substituted by halo, ⊚O or one or more heteroatoms;

provided X and A both are not H, and further provided that R⁵ is cyano or —CONHR¹ when A is H, halogen or NR¹R²;

or a compound having formula (TA6-1A)

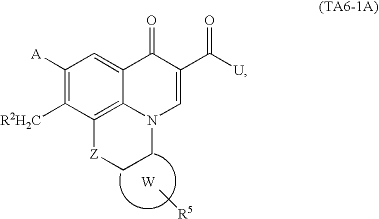

and pharmaceutically acceptable salts, esters and prodrugs thereof;

A is H, halogen, azido, SR², OR², CH₂R², NR¹R², or NR¹—(CR¹₂)ₙ—NR³R⁴;

Z, U, W, R¹, R², R³ and R⁴ are as defined in formula TA6-1; and

R⁵ is a substituent at any position of W and is H, halo, cyano, azido, —CONHR¹, OR², or C₁₋₆ alkyl or C₂₋₆ alkenyl, each optionally substituted by halo, ═O or one or more heteroatoms;

wherein each optionally substituted moiety in formula TA6-1 and -1A is substituted with one or more halo, cyano, azido, acetyl, amido, OR², NR¹R², carbamate, C₁₋₁₀ alkyl, C₂₋₁₀ alkenyl, each optionally substituted by halo, ═O, aryl or one or more heteroatoms selected from N, O and S; or is substituted with an aryl, a carbocyclic or a heterocyclic ring.

In the above formula TA6-1 or TA6-1A, W may be selected from the group consisting of

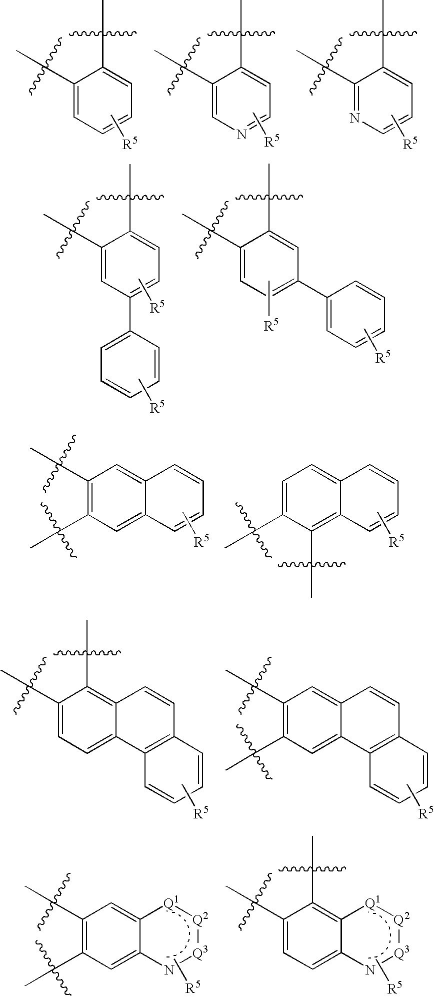

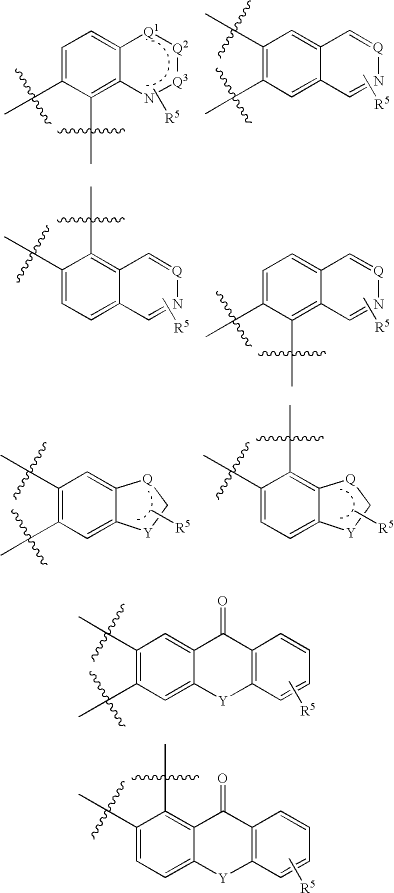

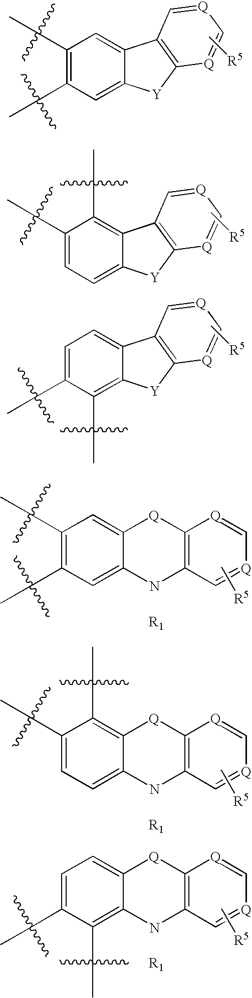

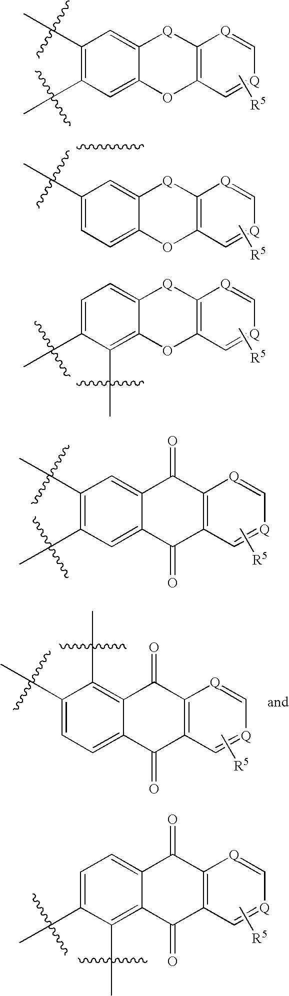

wherein Q, Q¹, Q², and Q³ are independently CH or N;

Y is independently O, CH, ═O or NR¹; and

R⁵ is as defined in formula 1.

In some embodiments of these compounds, each W in the above formula TA6-1 or TA6-1A may be an optionally substituted phenyl, pyridine, biphenyl, naphthalene, phenanthrene, quinoline, isoquinoline, quinazoline, cinnoline, phthalazine, quinoxaline, indole, benzimidazole, benzoxazole, benzthiazole, benzofuran, anthrone, xanthone, acridone, fluorenone, carbazolyl, pyrimido[4,3-b]furan, pyrido[4,3-b]indole, pyrido[2,3-b]indole, dibenzofuran, acridine or acridizine. In one embodiment, W is an optionally substituted phenyl.

The compounds of formula (TA6-1), and methods for making and using them, are described in U.S. patent application Ser. No. 11/404,947, to Whitten, et al., which was filed on Apr. 14, 2006, and is entitled QUINOBENZOXAZINE ANALOGS AND METHODS OF USING THEREOF.

A compound of general formula TA1-1A is a preferred therapeutic agent for use in the methods and compositions of the invention. More detail on methods for its formulation and administration are provided in U.S. provisional application Ser. No. 11/757,273, filed Jun. 1, 2007, naming Lim et al., and entitled DRUG ADMINISTRATION METHODS.

Compounds that can be delivered to reduce proliferation of cells involved in a disorder described herein can include compounds having significant effects on cell viability. Compounds described herein can be screened in a suitable assay to determine effects of the compound on cell viability. An example of such an assay involves the use of the dye Alamar Blue. Cells are counted using a hemocytometer, and 4,000-5,000 cells (per well) in 100 microliter of medium are seeded into wells 96-well plate. Cells are contacted with a compound described herein for a period of time. Twenty microliters of Alamar Blue reagent (stored at 4° C.) then is added to each well and the cells are incubated for four (4) hours at 37° C., 5% CO₂ in a humidified incubator. Fluorescence is recorded at an excitation wavelength of 544 nm and emission wavelength of 590 nm using a microplate reader. Fluorescence of non-reduced dye is detected and effects of drug treatment on cell viablity is determined.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

The compounds of the invention often have ionizable groups so as to be capable of preparation as salts. In that case, wherever reference is made to the compound, it is understood in the art that a pharmaceutically acceptable salt may also be used. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art. In some cases, the compounds may contain both an acidic and a basic functional group, in which case they may have two ionized groups and yet have no net charge.

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed. The compounds of the invention may also exist in more than one tautomeric form; the depiction herein of one tautomer is for convenience only, and is also understood to encompass other tautomers of the form shown.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain 1-10C (alkyl) or 2-10C (alkenyl or alkynyl). Preferably they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

“Acetylene” substituents are 2-10C alkynyl groups that are optionally substituted, and are of the formula —C≡C—R^{a}, wherein R^{a} is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl, and each R¹ group is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O; and

wherein two R′ can be linked to form a 3-7 membered ring optionally containing up to three heteroatoms selected from N, O and S. In some embodiments, R^{a} of —C≡C—R^{a} is H or Me.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)- and —C(Me)₂- may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Halo”, as used herein includes fluoro, chloro, bromo and iodo. Fluoro and chloro are often preferred.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle” refers to a cyclic compound containing only carbon atoms in the ring, whereas a “heterocycle” refers to a cyclic compound comprising a heteroatom. The carbocyclic and heterocyclic structures encompass compounds having monocyclic, bicyclic or multiple ring systems.

As used herein, the term “heteroatom” refers to any atom that is not carbon or hydrogen, such as nitrogen, oxygen or sulfur.

Illustrative examples of heterocycles include but are not limited to tetrahydrofuran, 1,3 dioxolane, 2,3 dihydrofuran, pyran, tetrahydropyran, benzofuran, isobenzofuran, 1,3 dihydro isobenzofuran, isoxazole, 4,5 dihydroisoxazole, piperidine, pyrrolidine, pyrrolidin 2 one, pyrrole, pyridine, pyrimidine, octahydro pyrrolo[3,4b]pyridine, piperazine, pyrazine, morpholine, thiomorpholine, imidazole, imidazolidine 2,4 dione, 1,3 dihydrobenzimidazol 2 one, indole, thiazole, benzothiazole, thiadiazole, thiophene, tetrahydro thiophene 1,1 dioxide, diazepine, triazole, guanidine, diazabicyclo[2.2.1]heptane, 2,5 diazabicyclo[2.2.1]heptane, 2,3,4,4a,9,9a hexahydro 1H β carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or contain carbon bound to elements other than hydrogen (e.g., elemental carbon, carbon monoxide, carbon dioxide, and carbonate). Examples of inorganic substituents include but are not limited to nitro, halogen, azido, cyano, sulfonyls, sulfinyls, sulfonates, phosphates, and the like.

Conditions Associated with Aberrant Cell Proliferation

The invention in part provides methods for treating ophthalmic conditions, methods for inhibiting cell proliferation and methods for treating an opthalmic condition related to aberrant cell proliferation. For example, provided are methods of treating a cell proliferative condition in a subject, which comprises administering a thereapeutic agent described herein to a subject in need of a treatment for a cell proliferative disorder; whereby the therapeutic agent is administered in an amount effective to treat the cell proliferative condition. The subject may be a research animal (e.g., rodent, dog, cat, monkey), optionally containing a tumor such as a xenograft tumor (e.g., human tumor), for example, or may be a human.

“Ophthalmic condition” as used herein pertains to a disease of the eye. Common ophthalmic conditions include, but are not limited to, the following examples.

Carcinomas and Melanomas

Basal Cell Carcinoma is the most common malignant tumor of the eyelid. It may form a lump (nodular form), a lump with a central crater (nodular-ulcerative form), a lump with a cyst (cystic form), or grow like a plaque (morpheaform). The lower lid is the most frequent site. This tumor does not metastasize, but it can burrow deep into the orbit. Exposure to sun is a risk factor.

Choroidal Melanoma (Brown mass) is a malignant melanoma arising in the choroid and elevating the retina. Choroidal melanomas have black pigment and appear as brown to yellow masses. The tumor probably arises via malignant transformation of nevi, collections of melanocytes.

Iris Melanoma (Brown iris mass) is a rare lesion and difficult usually an intensely brown mass which is either a nevus or a melanoma. The likelihood of metastasis is very low if the melanoma is confined to the iris. If it also involves the ciliary body, it poses more of a threat. Nevi are common; melanomas are rare and do not usually grow very much.

Retinoblastoma is the most common intraocular tumor of childhood and can occur in one or both eyes. The tumor arises from primitive neural retina and usually grows forward into the vitreous cavity. About 60% of cases are detected when they cause the cat's eye reflex, 25% when a child's eyes go out of alignment (strabismus), and 15% when they cause a child to fail routine vision screening or to develop a red eye or proptosis.

Central Retinal Artery Occlusions

Retinal edema is a condition in which the retina has become milky because of infarction. Tissue necrosis makes the tissue lose its normal transparency.

Cherry-red spot disorders results from the fact that the fovea contains only the photoreceptor layer, which is spared because it is nourished by the choroidal circulation rather than the retinal circulation. A cherry-red spot can also occur in lysosomal enzyme disorders in which lipid builds up in retinal ganglion cells. Good examples are Tay-Sachs, Mucolipidosis Type 1 and 2, Multiple Sulfatase Deficiency, and Niemann-Pick Type A.

Central retinal artery occlusions arise from two mechanisms: embolism and thrombosis. The relative prevalence of each mechanism is unknown. Emboli may come from the ophthalmic or carotid artery, aortic arch, or heart. Local thrombosis is associated with any prothrombotic state, most commonly arteriosclerosis.

Central Retinal Vein Occlusions

Flame-shaped hemorrhages result from an increased venous back pressure and the hypoxia of venous stasis cause widespread retinal hemorrhages. The hemorrhages lie between the ganglion cell axons in the retina's most superficial layer.

Distended retinal veins occur when increased pressure due to a downstream occlusion cause retinal veins to swell almost twice in size.

Retinal vein occlusions are noted by loss of vision, often mixed with sparkles most commonly caused by systemic hypertension, hyperviscous and hypercoagulable states.

Other Ocular Conditions

Other ocular conditions include vitreous detachment, which occurs when the vitreous has pulled away from the retinal surface. Detachment is sometimes so vigorously that it pulls on the retinal photoreceptors causing the patient to see a brief flurry of flashing lights. Vitreous detachment is a normal phenomenon of aging occurring after age 60, or earlier in high myopia, intraocular inflammation, hemorrhage, trauma or surgery. Retinal breaks also can result from tugging by a contracting vitreous. Other causes are degenerative disorders of the peripheral retina, ocular trauma and intraocular surgery. Retinal breaks can lead to retinal detachment. Retinal detachment generally occurs when a break in the retina allows vitreous fluid to seep underneath the retina and detach it. Common causes are aging, trauma, inflammation, high myopia, and intraocular surgery.

Age-Related Macular Degenerative Conditions

Macular Degeneration is a damage to the macula that occurs in some people as a natural process of aging. The most common form of macular degeneration is called “dry type”. Associated with aging it is caused by a breakdown of the tissues of the macula. Approximately 10% of macular degeneration is called exudative or “wet” type. In this case, small blood vessels begin to grow abnormally behind the retina often leading to scarring and reduced vision. While the “dry” type often produces symptoms gradually over many years, the “wet” type can produce a sudden onset. These blood vessels are very delicate and leak into the retina.

Retinal Drusen is noted by discrete orange blobs in the foveal region that signify death of the retinal pigment epithelium (RPE). Drusen arises as hereditary degenerations in young people, however the most common cause is age-related macular degeneration (ARMD). If the drusen become confluent in the macula, visual acuity will fall, but rarely below 20/40. In a small percentage of patients with ARMD, new blood vessels will grow from the choroid into the submacular retina, and bleed. This bleeding distorts the photoreceptors and causes blurred and warped vision (“metamorphopsia”).

Submacular Hemorrhage is noted by a round, brownish-black, pimple-like elevation in the macular region, a sign of bleeding under the retina. Usually it comes from new blood vessels that have burrowed into the retina from the underlying choroid.

Submacular neovascularization The most common cause is age-related macular degeneration (ARMD). The first stage is degeneration of the retinal pigment epithelium and the appearance of scattered retinal drusen, yellowish spots deep in the retina. They are associated with only mild visual acuity loss. Second stage is marked by new vessel growth under the retina followed by bleeding and exudation from these vessels first distorting vision, later devastating it. Other common causes of submacular neovascularization are angioid streaks, ocular histoplasmosis, trauma, and uveitis.

Ischemic Optic Neuropathy Conditions

Swollen optic disc conditions occur when increased intracranial pressure (ICP) exists slowing down axoplasmic flow at the optic disc and nerve. The margins of the optic disc become fuzzy and there are cotton wool spots on the superior border. Optic disc margins blur when the disc tissue rises above the surface blurring its margins. Sometimes caused by a congenital optic disc anomaly or by optic disc edema, an acquired slowing of axoplasmic flow. Optic disc margin hemorrhages occurs when ischemic leakage of retinal vessels on the optic disc surface is observed. And cotton wool spots occurs when white spots appear on the disc surface reflecting an explosion of ischemic axons and leakage of their axoplasm. The causes are comparable to an infarction caused by microvascular occlusion, especially in chronic hypertension and sudden hypotension situations.

Ischemic optic neuropathy is a term used to designate infarction of the optic nerve. The usual cause is small vessel arteriosclerosis in a patient with hypertension.

Infarction of the optic nerve is also the typical finding in giant cell (temporal) arteritis, an autoimmune disorder affecting medium-sized arteries in patients aged over 60 years. They often have one or more symptoms from two complexes: 1) polymyalgia rheumatica: malaise, limb girdle aches, and poor appetite; and 2) external carotid hypoperfusion: headache, scalp tenderness, and pain on chewing. The disease is caused by inflammatory occlusion of blood vessels.

Retinopathy of Prematurity

Dragged optic disc occurs when the optic disc is being pulled temporally by a preretinal membrane. This membrane has formed because of recurrent retinal bleeding from new blood vessels growing in the peripheral retina. Retinopathy of prematurity in infants with birth weights of 2000 gms or less. The retinal vessels are immature and when exposed to room oxygen, they may sprout neovascular buds that bleed, scar, and eventually detach the retina.

Strabismus: Accommodative Esotropia

Strabismus is the term used to designate ocular misalignment. There are many, many causes. For example, one of the three cranial nerves (III, IV, VI) could be damaged. Some infants are born with excessive convergence, or congenital esotropia. Apart from the cosmetic blemish, strabismus in early childhood is a danger because the nonfixating eye will quickly become amblyopic, that is, lose visual acuity. Amblyopia is a kind of disuse atrophy of the visual connections. If left untreated, visual loss from amblyopia may be permanent.

Amblyopia is the loss of visual acuity owing to visual deprivation or suppression of a retinal image. It is a kind of disuse atrophy that occurs only within the first five years of life, when the visual connections are still being modeled. During that time, neural connections will dissipate if the fovea of one eye is not receiving a clear image of a viewed object. Fortunately, these connections can be restored if the fovea gets a clear image soon enough. Amblyopia arises in one of four settings: 1) strabismus, or ocular misalignment; 2) anisometropia, or marked discrepancy between the refractive errors of the two eyes; 3) a corneal or lens opacity that degrades the image quality on the retina; and 4) ptosis that blocks any image from reaching the retina.

Optic Neuritis is acute visual loss owing to demyelination of the optic nerve. It may be an isolated autoimmune condition or part of multiple sclerosis. Visual acuity is usually depressed, but there are no abnormalities in the eye or its surrounding tissues.

Refractive Disorders fall into two main categories, Emmetropia and Ametropia. An emmetropic eye is one that has no refractive error. It will focus on the retina the image of an object viewed from a distance of 20 feet or more (optical infinity). An ametropic eye is one that has a refractive error, a mismatch between its optical power and axial length. There are three kinds of refractive errors that fall under these categories: myopia, hyperopia, and astigmatism. Myopia is the eye's excessive refractive power causing light rays to focus in front of the retina. Hyperopia is the eye's insufficient refractive power causes light rays to focus behind the retina. Astigmatism is the eye's abnormal corneal curvature preventing a point focus on the retina. That is not all optical planes are in focus. Presbyopia describes the loss of the eye's accommodative ability with aging. This age-related loss of accommodation, or presbyopia, is a form of induced hyperopia.

Myasthenia Gravis (MG) is an autoimmune disease, meaning that the body seems to turn on itself, producing antibodies to destroy healthy tissue and is typically described as experiencing the sensation of having “tired” eyes that appear to become droopy or sleepy-looking as the day progresses. They also report fatigue in their limbs, face and jaw. They may have trouble breathing, talking, chewing or swallowing. MG patients have eye disorders related to their condition.

Optic Nerve Disorders

A variety of neuro-ophthalmic illnesses affect the optic nerve. These include: Optic neuropathy which is a condition that often affects the elderly and people with extensive arteriosclerosis. Optic neuritis which is an inflammation of the optic nerve causing blurred vision and even temporary blindness. This condition is sometimes associated with multiple sclerosis. And optic edema which is swelling of the optic disc caused by an increase in intracranial pressure (papilledema) or by infection, inflammatory conditions or other conditions that create pressure in this area of the eye.

Diabetic Retinopathy: Diabetic eye disease refers to a group of eye problems that people with diabetes may face as a complication of this disease. All can cause vision loss. These diseases include: Diabetic retinopathy, cataract, glaucoma.

Cataract is another word for lens opacity causing blurred vision by disturbing the coherence of light rays as they pass through the eye, so that there is no clear focus on the retina. The opacification can begin anywhere in the lens and is the most common cause of blindness in the developing world. The most common cause of cataract is simply the aging process. Senescence causes the lens protein to degenerate and lose its transparency. Other causes are inflammation, trauma, metabolic, and hereditary disorders.

Glaucoma is a disease where pressure in the eye is usually elevated and causes damage to the optic nerve. Whether the damage to the optic nerve occurs due to high pressure or changes in blood flow to the nerve is not completely understood. Genetics plays some role and a specific gene for glaucoma was recently discovered. Glaucoma comes in four forms: primary open-angle, secondary, congenital, and angle-closure. Primary open-angle glaucoma is by far the most common. Elevated intraocular pressure is a factor in causing damage to the optic nerve. In eyes with primary open-angle glaucoma, the meshwork is abnormal and blocks the normal outflow of aqueous fluid. In secondary glaucoma, damage to the meshwork is caused by trauma, inflammation, or blood. Congenital glaucoma is caused by a malformed meshwork. It presents in infancy as tearing, photophobia, red and enlarged eye, and cloudy cornea. Angle-closure glaucoma results when the iris root plugs the opening of the trabecular meshwork. It is more common in older adults who have small eyes.

Keratoconus is a condition where the cornea develops a distorted shape producing blurred vision. While a normal eye is shaped round like a basketball. With nearsightedness, the cornea is too large and with farsightedness, the eye is too short. With astigmatism, the eye is shaped more like a football rather than a basketball. In other words, it is curved more in one direction than in the direction 90 degrees away. This “football shaped” cornea is what produces the distortion with astigmatism. Associated with this change in corneal shape, is a thinning of the central part of the cornea which eventually produces a “bulge” in the center of the cornea.

Uveitis is an inflammation of the the uvea, the layer includes the iris, ciliary body, and the choroid. The uvea provides most of the blood supply to the retina. Causes of uveitis can include autoimmune disorders, infection, or exposure to toxins. The most common form of uveitis is anterior uveitis or iritis, which involves inflammation in the front part of the eye usually limited to the iris. The inflammation may be associated with autoimmune diseases such as rheumatoid arthritis or ankylosing spondylitis, but most cases occur in healthy people and do not indicate an underlying disease. The disorder may affect only one eye and is most common in young and middle-aged people. A history of an autoimmune disease is a risk factor.

Pars planitis is inflammation of the pars plana, a narrow area between the iris and the choroid. Pars planitis usually occurs in young men and is generally not associated with any other disease. However, there have been a few case reports of an association with Crohn's disease, and some experts suggest a possible association with multiple sclerosis.

Posterior uveitis or choroiditis affects the back portion of the uveal tract, and involves primarily the choroid. If the adjacent retina is also involved it is called chorioretinitis. Posterior uveitis may follow a systemic infection or occur in association with an autoimmune disease. The inflammation causes spotty areas of scarring on the choroids, retina and or macula that result in areas of vision loss.

Trachoma is an infectious disease of the eye caused by the bacterium Chlamydia trachomatis. The bacteria can be spread easily on an infected person's hands or clothing, or may be carried by flies that have come in contact with discharge from the eyes or nose of an infected person. Because trachoma is transmitted through close personal contact, it tends to occur in clusters, often infecting entire families and communities.

Strabismus, more commonly known as cross-eyed or wall-eyed, is a vision condition in which a person can not align both eyes simultaneously under normal conditions. One or both of the eyes may turn in, out, up or down. An eye turn may be constant (when the eye turns all of the time) or intermittent (turning only some of the time, such as, under stressful conditions or when ill).

Cell lines associated with ophthalmic conditions include but are not limited to: CCL-20.2 (CCL), CEPI-17-CL4 (CEPI), SRA01/04 (LECs), 92.1, SP6.5, MKT-BR, OCM-1, and UW-1, SDHCEC1 and SDHCEC2, WKD, ECACC, 93120839, HCECs, HC0597, R28, LEC, SIRC, CCL 60; ATCC, Manassas, Va., WiDR, CT26, VUP, TM3, TM5, OM-431.

Therapeutic agents described herein also may be utilized to treat a nasal or otic condition. Examples of nasal conditions include, but are not limited to, nasal furunculosis, infectious rhinitis, rhinitis, eosinophilia syndrome, vasomotor rhinitis, epistaxis, septal hematoma, nasal polyp and nasal tumor (e.g., papilloma, osteoma (frontal or ethmoid sinus), squamous cell carcinoma, adenocarcinoma, lymphoma and melanoma). Otic conditions include, but are not limited to, autoimmune inner ear disease, cholesteatoma, otosclerosis and tumors (basal cell cancer, squamous cell cancer, ceruminoma).

### Administration of Compositions

The invention also in part provides pharmaceutical compositions comprising at least one therapeutic agent within the scope of the invention as described herein, which optionally may be administered in combination with at least one other compound. The composition may comprise a diluent or other pharmaceutically acceptable excipients. The pharmaceutical composition may be administered in an amount effective to treat a condition associated with aberrant cell proliferation in a subject in need thereof. The subject may be a research animal (e.g., rodent, dog, cat, monkey), optionally containing a tumor such as a xenograft tumor (e.g., human tumor), for example, or may be a human.

The terms “treat” and “treating” as used herein refer to ameliorating, alleviating, lessening, and removing symptoms of a disease or condition. A candidate molecule or compound described herein may be in a therapeutically effective amount in a formulation or medicament, which is an amount that can lead to a biological effect, such as apoptosis of certain cells (e.g., cancer cells), reduction of proliferation of certain cells, or lead to ameliorating, alleviating, lessening, or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system. Examples of microorganism include but are not limited to virus, bacterium and fungus.

In certain embodiments, a therapeutic agent may treat a condition by specifically inhibiting proliferation of cells associated with the condition to be treated. “Specifically inhibiting” or “specifically targeting” as used herein refers to inhibiting proliferation of cells associated with the condition to be treated more than inhibiting proliferation of “normal” cells. An example of a compound that specifically inhibits cells associated with a cell proliferation condition is compound TA1-1A, which inhibits leukemia cells without inhibiting normal bone marrow cells.

Agents desribed herein can result in apoptosis, and can thereby inhibit cell proliferation by resulting in the death of proliferating cells. As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

The amount of the therapeutic agent, and optionally one or more combination agents, to be administered will vary with the route of administration, the condition of the subject, other treatments being administered to the subject, and other parameters. The therapeutic agents of the invention may, of course, cause multiple desired effects; and the amount of modulator to be used in combination with the therapeutic agent should be an amount that increases one or more of these desired effects.

For administration to animal or human subjects, the appropriate dosage of the therapeutic agent sometimes is 0.01-15 mg/kg, preferably 0.1-10 mg/kg. Dosage levels are dependent on the nature of the condition, drug efficacy, the condition of the patient, the judgment of the practitioner, and the frequency and mode of administration; however, optimization of such parameters is within the ordinary level of skill in the art.

Similarly, the dosage of another compound used in combination with the therapeutic agent sometimes is between about 0.01-15 mg/kg, and can be about 0.1-10 mg/kg. Another agent used in combination with a therapeutic agent described herein may be separately active for treating a cancer. For combination therapies described above, when used in combination with a therapeutic agent, the dosage of another agent sometimes will be two-fold to ten-fold lower than the dosage required when the other agent is used alone to treat the same condition or subject. Determination of a suitable amount of the agent for use in combination with a therapeutic agent is readily determined by methods known in the art.

Any suitable formulation of the therapeutic agent can be prepared for administration. Any suitable route of administration may be used, including but not limited to, intraocular and topical. Administration also can include oral, parenteral, intravenous, intramuscular, nasal, transdermal, topical and subcutaneous routes, and the like. Depending on the subject to be treated, the mode of administration, and the type of treatment desired—e.g., prevention, prophylaxis, therapy; the compounds are formulated in ways consonant with these parameters. The formulation often is prepared according to the selected route of administration as known by the person of ordinary skill in the art. Preparation of suitable formulations for each route of administration are known in the art. A summary of such formulation methods and techniques is found in Remington's Pharmaceutical Sciences, latest edition, Mack Publishing Co., Easton, Pa., which is incorporated herein by reference. The formulation of each substance or a combination of two or more substances will generally include a diluent as well as, in some cases, adjuvants, buffers, preservatives and the like. Thus, provided herein are pharmaceutical compositions comprising a therapeutic agent and a pharmaceutically acceptable excipient. The substances to be administered can be administered also in liposomal compositions or as microemulsions.

Ophthalimic formulations can include aqueous solutions, aqueous suspensions, ointments, and inserts. Ophthalmic ointment bases often are a mixture of mineral oil and white petrolatum and have a melting point close to body temperature. Sometimes anhydrous lanolin is used to take up an ingredient that was dissolved in a small amount of water to affect dissolution. The aqueous solution is incorporated into the lanolin and then the lanolin is mixed with the remaining ointment base ingredients. Ointments generally are nonirritating and free from grittiness so the micronized form of the ingredients often is utilized. Sterile ointments are prepared by first sterilizing all of the individual ingredients and then combining them under aseptic conditions. The prepared ointment is then packaged in a sterile container such as an ointment tube. Ointments tend to blur patient vision as they remain viscous and are not removed easily by the tear fluid. Thus ointments are generally used at night as adjunctive therapy to eye drops used during the day. Ophthalmic ointment tubes are typically small holding approximately 3.5 g of ointment and fitted with narrow gauge tips which permit the extrusion of narrow bands of ointment.

Ocular inserts are not typically compounded and typically are manufactured. For example, Ocusert® is a nonerodible device designed to deliver pilocarpine for several days in the treatment of glaucoma. Some inserts are designed to dissolve in tear fluid. These inserts are made of dried polymeric solutions that have been fashioned into a film or rod. An example of this type of insert is Lacrisert® used to treat moderate to severe dry eye syndrome. Inserts generally are placed in the cul-de-sac between the eyeball and the eyelid. A disadvantage of inserts is their tendency to float on the eyeball, particularly in the morning upon arising.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised, and can be applied to compounds of the invention. See, for example, U.S. Pat. No. 5,624,677, the methods of which are incorporated herein by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

The therapeutic agent may be administered in conjunction with another agent, and the agents may be administered separately or together. When administered together, they may be in separate dosage forms, or they may be combined into a single combination drug.

A combination agent, when utilized, is administered in an amount that is effective to enhance a desired effect of the therapeutic agent. An amount is “effective to enhance a desired effect of the therapeutic agent”, as used herein, if it increases by at least about 25% at least one of the desired effects of the therapeutic agent alone. Preferably, it is an amount that increases a desired effect of the therapeutic agent by at least 50% or by at least 100% (i.e., it doubles the effective activity of the therapeutic agent.) In some embodiments, it is an amount that increases a desired effect of the therapeutic agent by at least 200%.

The amount of a combination agent that increases a desired effect of a therapeutic agent may be determined using in vitro methods, such as cell proliferation assays. The therapeutic agents of the invention are useful to counter hyperproliferative disorders such as cancer, thus they reduce cell proliferation. Thus, for example, a suitable amount of a combination agent could be the amount needed to enhance an antiproliferative effect of a therapeutic agent by at least 25% as determined in a cell proliferation assay.

The combination agent used in the present invention enhances at least one desired effect produced by the therapeutic agent it is used with, thus the combinations of the inveniton provide a synergistic effect, not merely an additive effect. The combination agents themselves are at times useful for treating the same types of conditons, and thus may also have some direct effect in such assays. In that event, the “amount effective to increase a desired effect” must be a synergistic enhancement of the activity of the therapeutic agent that is attributable to enhancement by the combination agent of an effect of the therapeutic agent, rather than a simple additive effect that would be expected with separate administration of the two materials. In many cases, the combination agent can be used in an amount (concentration) that would not be expected to have any apparent effect on the treated subject or the in vitro assay, so the increased effect achieved with the combination is directly attributable to a synergistic effect.

Some representative embodiments of the invention are set forth hereafter, but are not to be taken as limiting the scope of the invention as described herein.

A1. A method for inhibiting proliferation of cells by contacting an eye with a compound having a structure described herein.

A2. The method of embodiment Al, wherein the cells are cancer cells.

B1. A method for treating an ophthalmic disorder in a subject, which comprises administering to the subject a therapeutically effective amount of of a pharmaceutical composition comprising a compound having a structure described herein, whereby the disorder is treated.

B2. The method of embodiment B 1, wherein the disorder results from aberrant cell proliferation.

B3. The method of embodiment B2, wherein the disorder is a cancer.

B4. The method of any one of embodiments B1-B3, wherein the composition is administered to the eye of the subject.

B5. The method of embodiment B4, wherein the composition is administered topically.

C1. A method of any of the foregoing embodiments, wherein the composition comprises a compound of formula TA1-1.

C2. The method of embodiment Cl, wherein Z is O.

C3. The method of embodiment C2, wherein U is NR¹R².

C4. The method of embodiment C3, wherein U is NR¹R², wherein R² is H.

C5. The method of embodiment C4, wherein U is NR¹R², wherein R²is C₁₋₁₀ alkyl or C₂₋₁₀ alkenyl optionally substituted with a carbocyclic or heterocyclic ring.

C6. The method of embodiment C5, wherein X is NR¹R².

Citation of the above patents, patent applications, publications and documents herein is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following aspects.

What is claimed is:

1. A method for treating an ophthalmic, nasal, or otic disorder in a subject, which comprises administering to the subject a therapeutically effective amount of of a pharmaceutical composition comprising a compound having a structure selected from the group consisting of Formula TA1-1, Formula TA2-1, Formula TA3-1, Formula TA3-2, Formula TA4-1, Formula TA5-1, Formula TA6-1, and TA6-1A as described herein, whereby the disorder is treated.

2. The method of claim 1, wherein the disorder is an ophthalmic disorder.

3. The method of claim 2, wherein the ophthalmic disorder is a cancer.

4. The method of claim 3, wherein the ophthalmic disorder is selected from the group consisting of Carcinomas and melanomas, Central Retinal Artery Occlusions, Central Retinal Vein Occlusions, Other Ocular Conditions, Age-Related Macular Degenerative Conditions, Ischemic Optic Neuropathy Conditions, Retinopathy of Prematurity, Strabismus: Accommodative Esotropia, and Optic Nerve Disorders.

5. The method of claim 1, wherein the compound is a compound of Formula TA1-1: 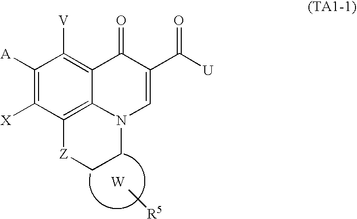
or a pharmaceutically acceptable salt or ester thereof;
wherein V is H, halo, or NR¹R²;
A is H, fluoro, or NR¹₂;
Z is O, S, NR¹ or CH₂;
U is OR² or NR¹R²;
X is OR², NR¹R², halo, azido, or SR²;
n is 1-3;
wherein in NR¹R², R¹ and R² may form a double bond or a ring, each of which is optionally substituted;
R¹ is H or a C₁₋₆ alkyl;
R² is H or a C₁₋₁₀ alkyl or C₂₋₁₀ alkenyl optionally containing one or more non-adjacent heteroatoms selected from N, O, and S, and optionally substituted with a carbocyclic or heterocyclic ring; or R² is an optionally substituted heterocyclic ring, aryl or heteroaryl;
R⁵ is a substituent at any position on W; and is H, OR², C₁₋₆ alkyl, C₂₋₆ alkenyl, each optionally substituted by halo, ═O or one or more heteroatoms; or R⁵ is an inorganic substituent; and
W is an optionally substituted aryl or heteroaryl, which may be monocyclic or fused with a single or multiple ring and optionally containing a heteroatom.

6. The method of claim 1, wherein the compound is a compound of Formula TA2-1 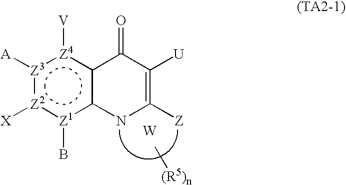
or a pharmaceutically acceptable salt or ester thereof;
wherein B, X, A, or V is absent if Z¹, Z², Z³, or Z⁴ respectively is N , and independently H, halo, azido, R², CH₂R², SR², OR² or NR¹R² if Z¹, Z², Z³, or Z⁴ respectively is C; or
A and V, A and X, or X and B may form a carbocyclic ring, heterocyclic ring, aryl or heteroaryl, each of which may be optionally substituted and/or fused with a cyclic ring;
Z is O, S, NR¹, CH₂, or C═O;
Z¹, Z², Z³ and Z⁴ are C or N, provided any three N are non-adjacent;
W together with N and Z forms an optionally substituted 5- or 6-membered ring that is fused to an optionally substituted saturated or unsaturated ring; said saturated or unsaturated ring may contain a heteroatom and is monocyclic or fused with a single or multiple carbocyclic or heterocyclic rings;
U is SO₃R², SO₂NR¹R², SO₂NR¹NR¹R², SO₂NR¹OR², SO₂NR¹—(CR¹₂)ₙ—NR³R⁴ or SO₂NR¹NR¹—(CR¹₂)ₙ—NR³R⁴ or SO₂NR¹—O—(CR¹₂)ₙ—NR³R;
in each NR¹R², R¹ and R² together with N may form an optionally substituted ring;
in NR³R⁴N, R³ and R⁴ together with N may form an optionally substituted ring; R¹ and R³ are independently H or C₁₋₆ alkyl; each R² is H, or a C₁₋₁₀ alkyl or C₂₋₁₀ alkenyl each optionally substituted with a halogen, one or more non-adjacent heteroatoms, a carbocyclic ring, a heterocyclic ring, wherein each ring is aryl or heteroaryl and optionally substituted; or R² is an optionally substituted carbocyclic ring or heterocyclic ring, wherein each ring is aryl or heteroaryl;
R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;
each R is a substituent at any position on ring W; and is H, OR², amino, alkoxy, amido, halogen, cyano or an inorganic substituent; or R⁵ is C₁₋₆ alkyl, C₂₋₆ alkenyl, C₂₋₆ alkynyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring that may be fused to an additional optionally substituted carbocyclic or heterocyclic ring; and
n is 1-6.

7. The method of claim 1, wherein the compound is a compound of Formula TA4-1: 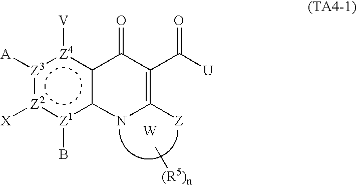
or a pharmaceutically acceptable salt or ester thereof;
wherein B, X, A, or V is absent if Z², Z³, or Z⁴, respectively, is N, and independently H, halo, azido, R², CH₂R², SR², OR² or NR¹R² if Z2, Z3, or Z⁴, respectively, is C; or
A and V, A and X, or X and B may form a carbocyclic ring, heterocyclic ring, aryl or heteroaryl, each of which may be optionally substituted and/or fused with a cyclic ring;
Z is O, S, NR¹, CH₂, or C═O;
Z¹, Z², Z³ and Z⁴ are C or N, provided any three N are non-adjacent;
W together with N and Z forms an optionally substituted 5- or 6-membered ring that is fused to an optionally substituted saturated or unsaturated ring; said saturated or unsaturated ring may contain a heteroatom and is monocyclic or fused with a single or multiple carbocyclic or heterocyclic rings;
U is R², OR², NR¹R, NR²—(CR¹₂)ₙ—NR³R⁴,or N═CR¹R², wherein in N═CR¹R²R¹ and R² together with C may form a ring,
provided U is not H, and when U is OH, OR² or NH₂, then at least one of Z¹-Z⁴ is N;
in each NR¹R², R¹ and R² together with N may form an optionally substituted ring;
in NR³R⁴, R³ and R⁴ together with N may form an optionally substituted ring; R¹ and R³ are independently H or C₁₋₆ alkyl; each R² is H, or a C₁₋₁₀ alkyl or C₂₋₁₀ alkenyl each optionally substituted with a halogen, one or more non-adjacent heteroatoms, a carbocyclic ring, a heterocyclic ring, wherein each ring is aryl or heteroaryl and optionally substituted; or R² is an optionally substituted carbocyclic ring, heterocyclic ring, wherein each eing is aryl or heteroaryl;
R⁴is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;
each R⁵ is a substituent at any position on ring W; and is H, OR², amino, alkoxy, amido, halogen, cyano or an inorganic substituent; or R⁵ is C₁₋₆ alkyl, C₂₋₆ alkenyl, C₂₋₆ alkynyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring that may be fused to an additional optionally substituted carbocyclic or heterocyclic ring; and
n is 1-6.

8. The method of claim 1, wherein the compound is a compound of Formula TA5-1: 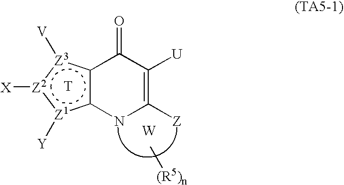
or a pharmaceutically acceptable salt or ester thereof;
wherein V, X, and Y are absent if attached to a heteroatom other than Nitrogen, and independently H, halo, azido, R², CH₂R², SR², OR² or NR¹R² when attached to C or N; or
wherein V and X, or X and Y may form a carbocyclic ring, heterocyclic ring, aryl or heteroaryl, each of which may be optionally substituted and/or fused with a cyclic ring;
Z¹, Z² and Z³ are C, N, O or S, wherein among Z¹, Z² and Z³ there is at most one O atom, among Z¹, Z² and Z³ there is at most one S atom, and among Z¹, Z² and Z³ there is at most two carbon atoms;
Z is O, S, NR, CH₂or C═O;
W together with N and Z forms an optionally substituted 5- or 6-membered ring that is fused to an optionally substituted aryl or heteroaryl, wherein said aryl or heteroaryl may be monocyclic or fused with a single or multiple ring, and wherein said ring optionally contains a heteroatom;
U is —C(═O)R², —COOR², —CONR¹R², —CONR¹—(CR¹₂)ₙ—NR³R⁴, SO₃R², SO₂NR¹R², SO₂NR¹NR¹R², SO₂NR¹OR², SO₂NR¹—(CR¹₂)ₙ—NR³R⁴ or SO₂NR¹NR¹—(CR¹₂)ₙ—NR³R⁴ or SO₂NR¹—O—(CR¹₂)ₙ—NR³R;
wherein in each NR¹R², R¹ and R² together with N may form an optionally substituted ring;
in NR³R⁴, R³ and R⁴ together with N may form an optionally substituted ring;
R¹ and R³ are independently H or C₁₋₆ alkyl;
each R₂ is H, or a C₁₋₁₀ alkyl or C₂₋₁₀ alkenyl each optionally substituted with a halogen, one or more non-adjacent heteroatoms selected from N, O and S, a carbocyclic ring, a heterocyclic ring, aryl or heteroaryl, wherein each ring is optionally substituted; or R² is an optionally substituted carbocyclic ring or heterocyclic ring, aryl or heteroaryl; or R² is COR¹ or S(O)ₓR¹ wherein x is 1-2;
R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;
each R⁵ is a substituent at any position on W; and is H, OR², amino, alkoxy, amido, halogen, cyano or an inorganic substituent; or R⁵ is C₁₋₆ alkyl, C₂₋₆ alkenyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring, optionally fused to an additional optionally substituted carbocyclic or heterocyclic ring; and
n is 1-6.

9. The method of claim 1, wherein the compound is a compound of Formula TA6-1: 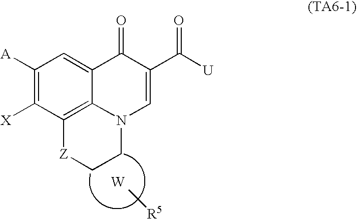
or a pharmaceutically acceptable salt or ester thereof;
wherein X is H, OR², NR¹R², halogen, azido, SR² or CH₂R;
A is H, halogen, NR¹R², SR², OR², CH₂R², azido or NR¹—(CR¹₂)ₙ—NR³R⁴;
Z is O, S, NR¹ or CH₂;
U is R², OR², NR¹R² or NR¹—(CR¹₂)ₙ—NR³R⁴ provided U is not H;
W is an optionally substituted aryl or heteroaryl, which may be monocyclic or fused with a single or multiple ring optionally containing a heteroatom;
wherein R¹ and R² together with N in NR¹R², and R³ and R⁴ together with N in NR³R⁴ may independently form an optionally substituted 5-6 membered ring containing N, and optionally O or S;
R¹ and R³ are independently H or a C₁₋₆ alkyl; and
R and R⁴ are independently H, or a C₁₋₁₀ alkyl or C₂₋₁₀ alkenyl optionally containing one or more non-adjacent heteroatoms selected from N, O, and S, and optionally substituted with a substituted or unsubstituted aryl, heteroaryl, carbocyclic, or heterocyclic ring; or R² is an optionally cycloalkyl, substituted heterocyclic ring, aryl or heteroaryl;
R⁵ is a substituent at any position of W and is H, halo, cyano, azido, —CONHR¹, OR², or C₁₋₆ alkyl or C₂₋₆ alkenyl, each optionally substituted by halo, ═O or one or more heteroatoms;
provided X and A both are not H, and further provided that R⁵ is cyano or —CONHR¹ when A is H, halogen or NR¹R²;
or a compound having formula (TA6-1A): 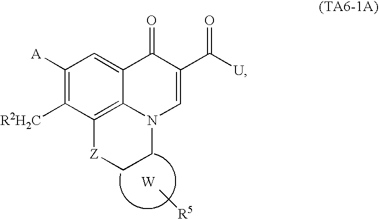
or a pharmaceutically acceptable salt or ester thereof;
A is H, halogen, azido, SR², OR², CH₂R², NR¹R², or NR¹—(CR¹₂)ₙ—NR³R⁴;
Z, U, W, R¹, R², R³ and R⁴ are as defined in formula TA6-1; and
R⁵ is a substituent at any position of W and is H, halo, cyano, azido, —CONHR¹, OR², or C₁₋₆ alkyl or C₂₋₆ alkenyl, each optionally substituted by halo, ═O or one or more heteroatoms;
wherein each optionally substituted moiety in formula TA6-1 and -1A is substituted with one or more halo, cyano, azido, acetyl, amido, OR², NR¹R², carbamate, C₁₋₁₀ alkyl, C₂₋₁₀ alkenyl, each optionally substituted by halo, ═O, aryl or one or more heteroatoms selected from N, O and S; or is substituted with an aryl, a carbocyclic or a heterocyclic ring.

10. The method of claim 1, wherein the composition is administered to the eye of the subject.

11. The method of claim 1, wherein the composition is administered topically.

12. The method of claim 10, wherein the composition is administered intraocularly.

13. The method of claim 1, wherein the composition comprises a compound of this formula: 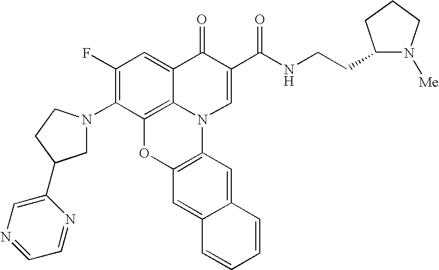
or a pharmaceutically acceptable salt thereof.

14. The method of claim 5, wherein Z is O.

15. The method of claim 14, wherein U is NR¹R².

16. The method of claim 15, wherein U is NR¹R², wherein R¹ is H.

17. The method of claim 16, wherein U is NR¹R², wherein R² is C₁₋₁₀ alkyl or C₂₋₁₀ alkenyl optionally substituted with a carbocyclic or heterocyclic ring.

18. The method of claim 17, wherein X is NR¹R².

19. A method to treat an ophthalmic disorder, comprising administering to a subject in need of such treatment an effective amount of a compound having a formula selected from the group consisting of Formula TA1-1, Formula TA2-1, Formula TA3-1, Formula TA3-2, Formula TA4-1, Formula TA5-1, Formula TA6-1, and TA6-1A.

20. The method of claim 19, wherein the ophthalmic disorder is selected from the group consisting of Central Retinal Artery Occlusions, Central Retinal Vein Occlusions, Other Ocular Conditions, Age-Related Macular Degenerative Conditions, Ischemic Optic Neuropathy Conditions, Retinopathy of Prematurity, Strabismus: Accommodative Esotropia, and Optic Nerve Disorders.

21. The method of claim 20, wherein the compound is of the formula 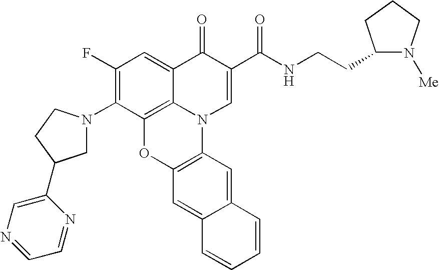
or a pharmaceutically acceptable salt thereof.
